(19) United States
(12) Patent Application Publication
(10) Pub. No.: US 2003/0138419 A1
(43) Pub. Date: Jul. 24, 2003

(54) Recombinant antibody fusion proteins and methods for detection of apoptotic cells

(75) Inventors: Marko Z. Radic, Germantown, TN (US); Brian A. Cocca, Silver Spring, MD (US)

Correspondence Address: JENKINS & WILSON, PA, 3100 TOWER BLVD, SUITE 1400, DURHAM, NC, 27707, US

(73) Assignee: The University of Tennessee Research Corporation

(21) Appl. No.: 10/295,757

(22) Filed: Nov. 15, 2002

Related U.S. Application Data
(60) Provisional application No. 60/332,193, filed on Nov. 16, 2001.

(51) Int. Cl. A61K 39/395; C07K 16/30

(52) U.S. Cl. 424/143.1; 530/388.22

(57) ABSTRACT

Recombinant antibody single chain variable fragments (scFv) useful for detecting apoptotic cells are disclosed. The antibodies selectively bind on the surface of apoptotic cells. Methods of generating and employing the antibodies are also provided. Methods of detecting modulation of apoptosis are disclosed. Methods of evaluating the efficacy of a candidate therapeutic compound adapted to effect a change in apoptosis are also disclosed. Additionally, a kit for the detection of apoptotic cells is also disclosed.

### CROSS REFERENCE TO RELATED APPLICATIONS

This application is based on and claims priority to U.S. Provisional Application Serial No. 60/332,193, filed Nov. 16,2001, herein incorporated by reference in its entirety.

### GRANT STATEMENT

This invention was made with Government support under Grant Al-34881 awarded by NIH. Therefore, the U.S. Government has certain rights in this invention.

### TECHNICAL FIELD

The present invention relates generally to antibodies and antibody fragments and to detection of apoptotic cells. More particularly, the present invention relates to recombinant antibody single chain variable fragments adapted to detect cells undergoing apoptosis. Additional applications of the recombinant single chain variable fragments are disclosed.

Abbreviations

| | |
|---|---|
| AEBSF | 1 mM 4-(2-aminoethyl) benzene-sulfonyl |
| | fluoride |
| BCA | bovine carbonic anhydrase |
| BSA | bovine serum albumin |
| CCD | cooled charge-coupled |
| CDR | complementarity determining region |
| DOPS | dioleoyl phosphatidylserine |
| ELISA | enzyme linked immunoabosrbent assay |
| FACS | fluorescence activated cell sorting |
| FBS | fetal bovine serum |
| FITC | fluoresceine isothiocyanate |
| Fv | variable fragment |
| HBSS | Hanks balanced salt solution |
| HSP | high scoring sequence pair |
| Ig | immunoglobulin |
| IgG | immunoglobulin G |
| MFI | mean fluorescent intensity |
| Ni-NTA | nickel N-(5-amino-1-carboxypentyl) |
| | iminodiacetic acid |
| PBS | phosphate buffered saline |
| PCD | programmed cell death |
| PCR | polymerase chain reaction |
| PI | propidium iodide |
| pl | isoelectric point |
| PNP | p-nitrophenol |
| PNPP | p-nitrophenol phosphate |
| RNP | ribonucleoprotein |
| scFv | single chain variable fragment |
| SDS-PAGE | sodium dodecylsuflate polyacrylamide gel |
| | electrophoresis |
| SLE | systemic lupus erythematosus |
| V_{H} | variable heavy chain |
| V_{L} | variable light chain |

Table of Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| Amino Acid | | | Codons |
|---|---|---|---|
| Alanine | Ala | A | GCA GCC GCG GCU |
| Cysteine | Cys | C | UGC UGU |
| Aspartic Acid | Asp | D | GAG GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA GUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BACKGROUND ART

I. Apoptosis (Programmed Cell Death)

Apoptosis, also referred to as Programmed Cell Death (PCD), is an essential regulator of tissue differentiation and cellular maintenance as animals develop and age (Saunders & Fallon, (1967) Cell Death in Morphogenesis, Major Problems in Developmental Biology; pp. 289-314, Academic Press, New York; Truman, (1984) Ann. Rev. Neurosci. 7: 171-188; Hurle, (1988) Meth. Achiev. Exp. Pathol. 13: 55-86; Ellis et al., (1991) Annu. Rev. Cell. Biol. 7: 663-698; Oppenheim, (1991) Ann. Rev. Neurosci. 14: 453-501; Raff, (1992) Nature 356: 397-400). Apoptosis is a cell suicide process of sequential biochemical events triggered by a variety of physiological and stress stimuli. Regulation of cell proliferation by apoptosis, maintains tissue homeostasis during development and differentiation (Raff, (1992) Nature 356:397-400; Vaux et al., (1994) Cell 76: 777-779).

Apoptosis involves an evolutionarly conserved multi-step cascade (Oltvai et al., (1994) Cell 79: 189-192), and is modulated by proteins that promote or counteract apoptotic cell death. Apoptosis also involves cell surface receptors (Smith et al., (1994) Cell 76, 959-962), and associated signal transducers (Tartaglia et al., (1992) Immunol. Today 13: 151-153), protease gene families (Martin et al., (1995) Cell 82: 349-352), intracellular second messengers (Kroemer et al., (1995) FASEB J. 9: 1277-1287), tumor suppressor genes (Hafifer et al., (1995) Curr. Opin. Genet. Dev. 5:84-90), and negative regulatory proteins that counteract apoptotic cell death (Hockenbery et al., (1990) Nature 348:334-336).

Several studies have implicated the misregulation of apoptosis in the pathophysiology of several human diseases including autoimmune disorders (e.g., SLE; see Walport, (2000) Nature Genet. 25: 135-136), atherosclerosis (Hofstra et al., (2000) Lancet 356: 209-212), AIDS (Meyaard et al., (1992) Science 257: 217-219; Gougen & Montagnier, (1993) Science 260: 1269-1270), neurodegenerative diseases (e.g., Alzheimer's disease and Tangiers disease) (Roy et al., (1995) Cell 80: 167-178; Liston et al., (1996) Nature 379: 349-353; Vito et al., (1996) Science 271: 521-525) and cancer (reviewed in Williams, (1991) Cell 65: 1097-1098; Steller, (1995) Science 267: 1445-1449; Thompson, (1995) Science 267: 1456-1462; Hall, (1999) Endocr.-Relat. Cancer 6: 3-8).

Several lines of evidence indicate that the physiology of apoptosis is quite highly conserved. First, the morphological changes associated with programmed cell deaths are similar in both vertebrates and invertebrates (Kerr et al., (1972) Br. J Cancer. 26: 239-257; Wyllie et al., (1980) Int. Rev. Cytol. 68: 251-306; Kerr & Harmon, (1991) in Apoptosis: The Molecular Basis of Cell Death, pp. 5-29, Cold Spring Harbor Laboratory Press, New York; Abrams et al., (1993) Development 117: 29-44). Second, at least two essential cell death genes in Caenorhabditis elegans, ced-3 and ced-9, are members of gene families that encode apoptotic functions in vertebrates (reviewed in Steller, (1995) Science 267: 1445-1449; White et al., (1996) Science 271: 805-807). Third, viral proteins that suppress apoptosis in their hosts (p35 and crmA) can exhibit potent anti-apoptotic activity in a wide range of heterologous species (Rabizadeh et al., (1993) J. Neurochem. 61: 2318-2321; Hay et al., (1994) Development 120: 2121-2129; Sugimoto et al., (1994) EMBO J 13: 2023-2028; Grether et al., (1995) Gene Dev. 9: 1694-1708; Pronk et al., (1996) Science 271: 808-810; White et al., (1996) Science 271: 805-807).

The process of apoptosis is distinguished from necrosis, another well-recognized form of cell death. Sudden anoxia, thermal extremes, or chemical toxicity can cause necrosis. Whole areas of tissue die after these injuries and individual cells have indistinct cytological appearances and disrupted membranes. Apoptotic cells, on the other hand, are decreased in size compared to their viable counterparts due to decreased cell water and loss of membrane-bound cytoplasmic blebs (Wyllie et al., (1980) Int. Rev. Cytol. 68: 251-306; Arends & Wyllie, (1991) Int. Rev. Exp. Pathol. 32: 223-549). The nuclei of apoptotic cells are homogeneously condensed and often fragmented. Internucleosomal double-stranded cleavage of nuclear DNA correlates closely with these nuclear morphological changes of apoptosis (Arends & Wyllie, (1991) Int. Rev. Exp. Pathol. 32: 223-549). Despite nuclear fragmentation and cytoplasmic blebbing, apoptotic cells retain their energy supply for an extended period of time and their plasma membranes remain intact (Wyllie et al., (1980) Int. Rev. Cytol. 68: 251; Arends & Wyllie, (1991) Int. Rev. Exp. Pathol. 32: 223-549).

In vivo, apoptosis occurs most commonly in individual cells that are scattered among non-apoptotic, normal neighbors. Specific molecules on the surface of the apoptotic cells lead to prompt recognition of these cells and subsequent phagocytosis by macrophages (Wyllie et al., (1980) Int. Rev. Cytol. 68: 251; Arends & Wyllie, (1991) Int. Rev. Exp. Pathol. 32: 223-549). This rapid removal of individual cells makes apoptosis much less apparent than necrosis, in vivo. Many chemotherapeutic agents used to treat acute leukemia induce apoptosis in vitro in leukemic cells lines and freshly isolated leukemic cells (Gunji et al., (1991) Cancer Res. 51: 741-743; Zwelling et al., (1993) Biochem. Pharmacol. 45: 516; Karp et al., (1994) Blood 83: 517-530; Campana et al., (1993) Leukemia 7: 482; Bhalla et al., (1992) Blood 80: 2883-2390; Miyashita & Reed, (1993) Blood 81:151-157; Lotem & Sachs, (1992) Blood 80: 1750-1757; Bhalla et al., (1993) Blood 82: 3133-3140; Chiron et al., (1992) Blood 80: 1307). Apoptosis has been demonstrated in the blood and bone marrow of patients receiving combined chemotherapy for acute leukemia (Li et al., (1994) Leukemia Lymphoma 13: 65). Thus, the measurement of apoptosis in vitro can provide a mechanism to assay for chemosensitivity of a purified leukemic cell population.

II. Structure and Features of Antibodies

The medical and research communities have exploited the interaction between antibodies and antigens for a variety of detection methodologies for over 30 years. Common techniques include tissue staining, radioimmunoassays, enzyme immunoassays, fluorescence immunoassays, and immunoblotting. In each case, the unique ability of an antibody to bind specifically to a particular antigen is exploited.

Structurally, an antibody has two functionally distinct regions, called the “variable” region, and the “constant” region, respectively. The variable region can bind to an antigen or epitope without the formation of covalent chemical bonds. The constant region can associate with cellular receptors. Differences in the molecular make-up of the constant regions define particular classes and subclasses of immunoglobulins. There are five principal classes, denoted in the art as IgG, IgA, IgM, IgD and IgE, with IgG being the most prevalent.

Native antibodies are synthesized primarily by specialized lymphocytes called “plasma cells.” Production of a strong antibody response in a host animal is controlled by inducing and regulating the differentiation of B cells into these plasma cells. This differentiation involves virgin B cells (which have a cell-surface-anchored antibody as an antigen receptor and do not secrete antibodies) becoming activated B cells (which both secrete antibodies and have cell-surface antibodies), then plasma cells (which are highly specialized antibody factories with drastically reduced surface antigen receptors). This differentiation process is influenced by the presence of antigen and by cellular communication between B cells and helper T cells.

Because of their ability to bind selectively to an antigen of interest, antibodies have been used widely for research, diagnostic and therapeutic applications. The potential uses for antibodies were expanded with the development of monoclonal antibodies. In contrast to polyclonal antiserum, which includes a mixture of antibodies directed against different epitopes, monoclonal antibodies are directed against a single determinant or epitope on the antigen and are homogeneous. Moreover, monoclonal antibodies can be produced in substantially unlimited quantities.

III. Available Apoptosis Detection Methods

Procedures to detect cell death based on the TdT-mediated dUTP nick end-labeling (TUNEL) method are commercially available from Roche (Cell Death Kit), Oncor (APOPTAG PLUS™) and Promega (DEADEND™). This method involves a number of limitations. Early detection of apoptosis is not possible with this method because the DNA ladder is an end-point in the apoptosis pathway. Also, although the TUNEL method distinguishes live cells from dead, it does not accurately determine whether the cells died by apoptosis or necrosis. False positives are often obtained when using the TUNEL method as a result of DNA fragments from cells that have died by necrosis: random DNA breakdown during necrosis generates DNA fragments that have 3′-OH ends. False negatives can also occur in certain cell types or situations where apoptosis does not lead to DNA laddering. Furthermore, the method is not quantitative since the amount of DNA fragments per cell is dependent upon the stage of apoptosis of the cell.

Another marker that is commercially available is annexin, sold under the trademark APOPTEST™, available from DAKO of Carpinteria, Calif. This marker is also used in the “Apoptosis Detection Kit” offered by R&D Systems of Minneapolis, Minn. During apoptosis, a cell membrane's phospholipid asymmetry changes such that a particular phospholipid, phosphatidylserine, becomes exposed on the outer membrane. Annexins are a homologous group of proteins that bind phosphatidylserine in the presence of calcium. A second reagent, propidium iodide (PI), is a DNA binding fluorochrome. When a cell population is exposed to both reagents, apoptotic cells stain positive for annexin and negative for PI, necrotic cells stain positive for both, live cells stain negative for both. This marker, however, suffers from a number of problems. Annexin has a strict requirement for Ca²⁺ for binding and may not detect apoptosis in all cell types (King et al., (2000) Cytometry 1: 10-18). Additionally, its use is limited to cells grown in suspension, and most cells are adherent and are grown on a matrix. The method also requires the use of live or unpreserved cells.

The present invention addresses these problems associated with methods of identifying apoptotic cells, as well as other problems. Thus, the present invention is a significant advance over prior art compositions and methods.

### SUMMARY OF THE INVENTION

An isolated antibody composition is disclosed. In one embodiment, the antibody composition comprises a 3H9 antibody-derived variable region that specifically recognizes an epitope on the surface of an apoptotic cell, the epitope being detectable in cells undergoing apoptosis and undetectable in cells not undergoing apoptosis

An isolated and purified polynucleotide encoding an antibody polypeptide is disclosed. In one embodiment, the polynucleotide comprising one or more of: a polynucleotide encoding a variable segment of a heavy chain of an antibody, a polynucleotide encoding a variable segment of a light chain of an antibody, a polynucleotide encoding a linker sequence, a polynucleotide encoding a dimerization domain, a selectable marker, a polynucleotide encoding a purification sequence and combinations thereof.

A method of identifying an apoptotic cell is disclosed. In one embodiment, the method comprises: contacting an antibody composition adapted to recognize an epitope on the surface of an apoptotic cell with a cell; and detecting association of the antibody composition with the epitope, the association being indicative of an apoptotic cell.

A method of evaluating the efficacy of a candidate therapeutic compound adapted to effect a change in apoptosis is disclosed. In one embodiment, the method comprises: (a) contacting an antibody composition adapted to recognize an epitope on the surface of an apoptotic cell with a first sample comprising cells capable of apoptosis; (b) quantifying an extent to which apoptosis is occurring in the first sample; (c) contacting a candidate therapeutic with a second sample comprising cells capable of apoptosis; (d) contacting the antibody composition with the second sample; (e) quantifying a second degree to which apoptosis is occurring; and (f) comparing the first and second degrees of apoptosis, whereby the efficacy of a candidate therapeutic compound adapted to effect a change in apoptosis is evaluated.

A kit for detecting apoptotic cells is disclosed. In one embodiment, the kit comprises an antibody composition that specifically recognizes an epitope on the surface of an apoptotic cell; a cell culture medium; and a detection reagent adapted to indicate the presence of an immunocomplex comprising an antibody composition and an apoptotic cell.

A method of screening a population of antibodies to identify an antibody adapted to detect cells undergoing apoptosis is disclosed. In a one embodiment, the method comprises: (a) providing a library comprising one of a population of diverse antibodies and a phage display library comprising an antibody fusion protein to be screened; (b) contacting the library with a population of cells comprising apoptotic cells to thereby form a mixture; (c) contacting the mixture with a 3H9-derived antibody composition adapted to specifically recognize an epitope on the surface of an apoptotic cell, the epitope being detectable in cells undergoing apoptosis and undetectable in cells not undergoing apoptosis to thereby form a detection mixture comprising bound antibodies; (d) contacting the detection mixture with a detectably labeled antibody adapted to recognize the 3H9-derived antibody composition, thereby identifying the presence of apoptotic cells; and (e) separating apoptotic cells from non-apoptotic cells; and (f) eluting bound antibodies.

An isolated antibody composition is disclosed and, in one embodiment, specifically recognizes an epitope on the surface of an apoptotic cell, the epitope being present in a complex comprising phosphatidylserine, dioleoyl phosphatidylserine, β2GPI, a nucleoprotein (e.g., a histone), a constituent of an apoptotic cell surface and combinations thereof, and being detectable in cells undergoing apoptosis and undetectable in cells not undergoing apoptosis.

Accordingly, it is an object of the present invention to provide an antibody composition adapted to specifically recognize an epitope on the surface of an apoptotic cell, the epitope being detectable in cells undergoing apoptosis and undetectable in cells not undergoing apoptosis. This and other objects are achieved in whole or in part by the present invention.

Some of the objects of the invention having been stated hereinabove, other objects will be evident as the description proceeds, when taken in connection with the accompanying drawings as best described hereinbelow.

### BRIEF DESCRIPTION OF THE DRAWINGS

FIG. 1 is a ribbon diagram depicting the 3H9 combining site highlighting sidechains exchanged by mutagenesis (isoleucine 57 (I57), aspartic acid 65 (D65), and arginine 53 (R53) were reverted to germline, and individual arginine residues were introduced at positions 31 (R31), 56 (R56), 58 (R58) and 76 (R76)).

FIGS. 2A and 2B are plots depicting the results of DOPS binding in solid phase-ELISA (FIG. 2A summarizes results for 3H9 (●) and its revertant variants: RS3G (▪), I57T (▾), D65G (▴), and R53G/I57T/D65G (♦); FIG. 2B summarizes results for 3H9 with forward mutations to arginine: S31 R (∘), D56R (□), N58R (Δ), S76R (⋄), and D56R/S76R (∇)).

FIGS. 2C and 2D are plots depicting the results of a binding assay comprising an scFv and DOPS complexed with β2GPI. (FIG. 2C summarizes results for 3H9 (●) and its revertant variants: RS3G (▪), I57T (▾), D65G (▴), and R53G/I57T/D65G (♦); FIG. 2D summarizes results for 3H9 with forward mutations to arginine: S31R (∘), D56R (□), N58R (Δ), S76R (⋄), and D56R/S76R (∇)).

FIG. 3 is a plot depicting inhibition of DOPS-β2GPI binding by DNA or DOPS-β2GPI vesicles (D56R/S76R (□, ▪) or R53G/I57T/D65G (∘, ●), were incubated with increasing concentrations of DNA (open symbols) or DOPS-β2GPI vesicles (filled symbols) prior to incubation on DOPS-β2GPI-coated ELISA plates).

FIG. 4A is a flow cytometric analysis of scFv binding to staurosporine-treated Jurkat cells which were gated according to forward and side scatter to exclude cell fragments and debris.

FIG. 4B is a flow cytometric analysis of scFv binding to staurosporine-treated Jurkat cells which were gated according to forward and side scatter to exclude cell fragments and debris and were further gated into annexin V-positive and -negative populations.

FIG. 4C is a flow cytometric analysis of Annexin V-positive and -negative cells indicating the extent of scFv binding and PI staining (Annexin V-positive cells (left column) are bound by D56R/S76R and R53G/I57T/D65G (germline), although some Annexin V-positive cells fail to bind scFv and no binding of scFv to Annexin V-negative cells was detected (right)).

FIG. 5 is a polyacrylamide gel depicting the purification of scFv by Ni-NTA affinity chromatography (Lanes are marked as follows: MW, molecular weight marker; lane 1, R53G; lane 2, 157T; lane 3, D65G; lane 4, R53G/I57T/D65G; lane 5, 3H9; lane 6, S31 R; lane 7, D56R; lane 8, N58R; lane 9, S76R; lane 10, D56R/S76R).

FIG. 6A is a plot depicting the binding of D56R/S76R (▪) and 3H9/62.1 (●) to DOPS-β2GPI in ELISA.

FIG. 6B is a plot depicting the binding of D56R/S76R (▪) and 3H9/62.1 (●) to double stranded DNA in ELISA.

FIG. 7 is a flow cytometric analysis comparing Annexin V and D56R/S76R binding to apoptotic cells treated with staurosporine, camptothecin, or anti-Fas to induce apoptosis. The scFv bound only to annexin V-positive cells and binding could be blocked by Z-VAD-FMK, an inhibitor of apoptosis.

FIG. 8 is a fluorescence microscopy picture of an apoptotic Jurkat cell showing binding of scFv to apoptotic blebs. Binding of scFv and annexin V is largely segregated, in that annexin V binds between blebs. Most blebs bound by the scFv contain pieces of the fragmented nucleus that are stained by TO-PRO3, a DNA binding dye.

### BRIEF DESCRIPTION OF THE SEQUENCES IN THE SEQUENCE LISTING

SEQ ID NO: 1 is a 15-mer amino acid sequence comprising a linker sequence of the present invention.

SEQ ID NO: 2 is a nucleic acid sequence of a vector comprising an scFv of the present invention.

SEQ ID NO: 3 is a PCR primer that can be employed in the present invention.

SEQ ID NO: 4 is a PCR primer that can be employed in the present invention.

SEQ ID NO: 5 is a PCR primer that can be employed in the present invention.

SEQ ID NO: 6 is a nucleotide sequence encoding a R53G/I57T/D65G scFv mutant of the present invention.

SEQ ID NO: 7 is a nucleotide sequence encoding a D56R/S76R scFv mutant of the present invention.

SEQ ID NO: 8 is a nucleotide sequence encoding a V_{L} chain identified by Genbank Accession Number X17634.

SEQ ID NO: 9 is a nucleotide sequence encoding a V_{L} chain identified by Genbank Accession Number U29768.

SEQ ID NO: 10 is a nucleotide sequence encoding a V_{L} chain identified by Genbank Accession Number U29780.

SEQ ID NO: 11 is a nucleotide sequence encoding a V_{L} chain identified by Genbank Accession Number U30232.

### DETAILED DESCRIPTION OF THE INVENTION

The present invention comprises, in part, an antibody composition adapted to recognize an epitope present on the surface of an apoptotic cell. The antibody composition can thus discriminate between apoptotic and viable (or necrotic) cells. It is notable that the epitope recognized by the antibody composition is disposed on the surface of the cell. This represents a significant advantage over prior art methods of identifying apoptotic cells. These prior art methods require a recognizable epitope to be disposed on a structure on the interior of an apoptotic cell, such as the mitochondrion. Thus, prior art methods require that cells be lysed in order to expose the, epitope, after which it can be determined whether or not the cells are apoptotic cells. The present invention solves this problem by providing an antibody composition adapted to recognize a surface epitope, thereby eliminating the need to lyse the cells to assess apoptosis.

In one embodiment, the antibody composition comprises an scFv. Thus in, another aspect of the present invention, an scFv of the present invention can be expressed in a bacterial system. This also represents an advance over the prior art, because there is no need to prepare a hybridoma or other complex system to express an scFv of the present invention. Conversely, an scFv of the present invention can be expressed in a convenient bacterial system and can be purified by employing standard protein purification methods. Additionally, the ability to employ a bacterial expression system facilitates the ability to readily prepare scFv mutants, chimeras and fusion proteins.

The present invention facilitates screening cells for apoptotic cells. Thus, a population of cells can be screened and apoptotic cells, as well as viable cells, can be identified. Additionally, the ability to easily prepare an antibody composition of the present invention also facilitates high throughput screening of a candidate therapeutic adapted to modulate apoptosis. This can be desirable when the apoptosis is associated with a disease condition.

I. Definitions

Following long-standing patent law convention, the terms “a” and “an” mean “one or more” when used in this application, including the claims.

As used herein, the term “antibody” is used in its broadest sense and specifically covers monoclonal antibodies (including agonist, antagonist, and blocking or neutralizing antibodies) and antibodies with polyepitopic specificity. It is emphasized that the term “antibody” encompasses not only “complete” antibodies (i.e. antibodies comprising an Fc region and two Fab regions, such as intact IgG, IgE, IgM, IgA and IgD antibodies, and variants thereof), but also fragments thereof. Thus, the term encompasses any composition retaining the ability to recognize one or more epitopes. Therefore, the term “antibody” encompasses monomeric, dimerized or polymeric single chain variable fragment (scFv) polypeptides and fusion proteins between scFv and other functional domains, such as those produced by the recombinant methods of the present invention.

As used herein, the term “antibody composition” means a composition comprising an antibody or an antibody fragment.

The terms “apoptosis” and “apoptotic activity” are used in their broadest sense and refer to the orderly or controlled form of cell death in mammals and other vertebrates, and some invertebrates as well. The morphological features of apoptosis include an orchestrated sequence of changes which include cell shrinkage, loss of plasma membrane microvilli, bleb formation, chromatin condensation, loss of mitochondrial function, nuclear segmentation and eventual cellular disintegration into discrete membrane-bound apoptotic bodies. The biochemical features include, for example, internucleosomal cleavage of cellular DNA. This activity can be determined and measured, for instance, by cell viability assays, FACS analysis or DNA electrophoresis, all of which are known in the art.

As used herein, the terms “single-chain Fv” and “scFv” are used interchangeably and mean a polypeptide comprising the V_{H} and V_{L} domains of antibody, wherein these domains are connected by a polypeptide linker between the V_{H} and V_{L} domains into a single polypeptide chain. The linker enables the scFv to form the desired structure for epitope binding. For a review of scFv proteins see, e.g., Pluckthun, (1994) The Pharmacology of Monoclonal Antibodies, vol. 113, (Rosenburg and Moore, eds.), Springer-Verlag, New York, N.Y., pp. 269-315. An scFv can also comprise a dimerization domain, facilitating the formation of scFv dimers. Thus, when referring to an scFv, the term is intended to also refer to scFv dimers, even though scFv dimers might not be explicitly enumerated.

As used herein, the terms “variant” and “antibody variant” are used interchangeably and mean a biologically active polypeptide having at least about 80% amino acid sequence identity over the length of a V_{H} or a V_{L} sequence. Such variants include, for instance, polypeptides wherein one or more amino acid residues are added, or deleted, at the N- or C-terminus of the polypeptide. Ordinarily, a variant will have at least about 80% amino acid sequence identity, more preferably at least about 90% amino acid sequence identity, and even more preferably at least about 95% amino acid sequence identity with native sequence.

Thus, the term “variant” means an amino acid sequence, particularly an amino acid sequence of the present invention, which is altered by one or more amino acids. As noted further herein, the variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Analogous minor variations can also include amino acid deletions or insertions, or both. Guidance in determining which amino acid residues can be substituted, inserted, or deleted without abolishing biological or immunological activity can be found using computer programs known to those of skill in the art. Additional guidance is provided herein below. The term “variant” is used interchangeably with the term “mutant”.

As used herein, the terms “sequence identity”, “percent (%) sequence identity” and “percent (%) identity” are used interchangeably and are defined as the percentage of amino acid residues in a candidate sequence that are identical with the amino acid residues in the native sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as ALIGNT or Megalign (DNASTAR) software. Those skilled in the art can determine appropriate parameters for measuring alignment, including any algorithms needed to achieve maximal alignment over the full length of the sequences being compared.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, blastoma, gastrointestinal cancer, renal cancer, pancreatic cancer, glioblastoma, neuroblastoma, cervical cancer, ovarian cancer, liver cancer, stomach cancer, bladder cancer, breast cancer, colon cancer, colorectal cancer, endometrial carcinoma, salivary gland carcinoma, kidney cancer, liver cancer, prostate cancer, vulval cancer, thyroid cancer, hepatic carcinoma and various types of head and neck cancer.

The term “mammal” as used herein refers to any animal classified as a mammal, including humans, cows, horses, mice, rats, dogs and cats.

As used herein, the terms “mutation” and “mutant” carry their traditional connotations and means a change, inherited, naturally occurring or introduced, in a nucleic acid or polypeptide sequence, and is used in its sense as generally known to those of skill in the art.

As used herein, the term “isolated”, when referring to a polypeptide, means a polypeptide (which can comprise an antibody) that has been identified and separated and/or recovered from a component of its natural environment. Contaminant components of its natural environment are materials that would interfere with diagnostic or therapeutic uses for the polypeptide, and can include enzymes, hormones, and other proteinaceous or nonproteinaceous solutes. In some embodiments, the polypeptide will be purified (1) to greater than 95% by weight of polypeptide as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or nonreducing conditions and employing Coomassie blue or silver stain.

As used herein, the term “isolated”, when referring to a nucleic acid molecule means a nucleic acid molecule that is identified and separated from at least one contaminant nucleic acid molecule with which it is ordinarily associated in the natural source of the nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the polypeptide where, for example, the nucleic acid molecule is in a chromosomal location different from that of natural cells.

As used herein, the terms “cell,” “cell line,” and “cell culture” are used interchangeably, and all such designations include progeny. Thus, the words “transformants” and “transformed cells” include the primary subject cell and cultures derived therefrom without regard for the number of transfers. It is also understood that all progeny might not be precisely identical in DNA content, due to deliberate or inadvertent mutations. Mutant progeny that have the same function or biological activity as screened for in the originally transformed cell are encompassed by the terms. Where distinct designations are intended, it will be clear from the context.

As used herein, the term “antigen” means a region or regions of a structure (e.g., a protein, nucleic acid, carbohydrate or lipid) or fragment of a structure (e.g., a protein fragment) that can be employed to immunize a host and/or that can elicit antibody formation. Numerous regions of the structure can induce the production of antibodies which bind specifically to a given region or three-dimensional structure on the structure; these regions or structures are referred to as “antigenic determinants”.

As used herein, the term “epitope” means a particular structure on an antigen that is recognized by an antibody. Thus, a single antigen can comprise a plurality of epitopes. Generally, the term “epitope” means an arrangement of atoms on an antigen that is bound by an antibody

As used herein, the term “polymerase chain reaction” (PCR) means the method of Mullis and embodied, in part, in U.S. Pat. Nos. 4,683,195 and 4,683,202, hereby incorporated by reference, which describe a method for increasing the concentration of a segment of a target sequence in a mixture of genomic DNA without cloning or purification.

As used herein, the terms “PCR product” and “amplification product” mean the resultant mixture of compounds after two or more cycles of the PCR steps of denaturation, annealing and extension are complete. These terms encompass the case where there has been amplification of one or more segments of one or more target sequences.

As used herein, the term “amplification reagents” means those reagents (deoxyribonucleoside triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonuclease” and “restriction enzyme” means a bacterial enzyme, which is adapted to cut double-stranded DNA at or near a specific nucleotide sequence.

As used herein, the term “recombinant DNA molecule” means a DNA molecule that is comprised of segments of DNA joined together by means of molecular biological techniques and that is capable of propagation in a host organism or in vitro.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also might be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “epitope of the present invention” means an epitope on the surface of an apoptotic cell, the epitope being present in a complex comprising phosphatidylserine, dioleoyl phosphatidylserine, β2GPI, a nucleoprotein (e.g., a histone), a constituent of an apoptotic cell surface and combinations thereof and being detectable in cells undergoing apoptosis and undetectable in cells not undergoing apoptosis.

As used herein, the term “nucleoprotein” means a protein that can associate with a nucleic acid or nucleic acid sequence (e.g., a histone). The term also encompasses a complex comprising a protein that can associate with a nucleic acid or nucleic acid sequence and a nucleic acid or nucleic acid sequence (e.g., a histone associated with a DNA sequence). Thus, as the term is employed herein, a “nucleoprotein” comprises a complex comprising a protein that can associate with a nucleic acid as well just as a protein that is capable of associating with a nucleic acid or nucleic acid sequence, with no nucleic acid or nucleic acid sequence bound to the protein.

II. General Considerations

In view of the biological importance of apoptosis, there exists a need for methods to specifically detect cells undergoing apoptosis and those that have suffered apoptotic cell death. These methods are crucial to the identification, characterization, and diagnosis of diseases distinguished by abnormal apoptosis, and to the screening of potential therapeutic agents that can induce or prevent apoptosis. Techniques for detection of apoptosis can also be employed to screen for candidate therapeutic agents that can induce, prevent or modulate apoptosis.

Several methods are known for the detection of apoptosis in vitro and in vivo, but these methods have significant drawbacks, which limit their utility. Apoptosis is characterized, in one aspect, by condensation and margination of nuclear chromatin, and fragmentation of nuclear structure into so-called apoptotic bodies. This apoptotic morphology can be observed using conventional stains, dyes which selectively accumulate in nuclei such as propidium iodide or Hoechst 33258, or by electron microscopy (e.g., Nicoletti et al., (1991) J. Immunol. Methods 139: 271-279; Crompton et al., (1992) Biochem. Biophys. Res. Commun. 183: 532-537; Frey, (1995) Cytometry 21: 265-274 1995; Woo, (1995) N. Engl. J. Med. 333: 18-25). Unfortunately, these techniques are either of insufficient specificity or are too laborious and technically complex for the routine selective identification and quantification of apoptotic cells in situ.

Recent attempts to identify and quantify apoptosis have taken advantage of the internucleosomal fragmentation of DNA, which is often linked to, but is not diagnostic for, cell death by apoptosis. Various in situ histochemical techniques have been applied to the end-labeling of nicked DNA (Gavrieli et al., (1992) J. Cell Biol. 119: 493-501; Wijsman et al., (1993) J. Histochem. Cytochem. 41: 7-12; Wood et al., (1993) Neuron 11: 621-632). Although these techniques have become popular for marking apoptotic cells in situ, it has been recognized, as noted hereinabove, that DNA fragmentation can also result from cell stress or necrotic degeneration. Consequently, the in situ techniques that detect fragmented DNA are not fully selective in detecting cells undergoing apoptosis (Nitatori et al., (1995) J. Neurosci. 15: 1001-1011; Lassmann et al., (1995) Acta Neuropathol. 89: 35-41).

Molecular techniques have also been employed for the detection in cell and tissue extracts of internucleosomal DNA degradation linked to apoptosis (Wyllie, (1980) Nature 284:555-556; Wyllie et al., (1984) J. Pathol. 142:67-77). In situ and molecular techniques that rely on the detection of internucleosomal DNA fragmentation are not sufficiently thorough for the detection of apoptotic cell death, since they do not detect forms of apoptosis not associated with internucleosomal DNA degradation (Cohen et al., (1992) Biochem. J. 286:331-334; Schulze-Osthoff et al., (1994) J. Cell Biol. 127:15-20). Moreover, the molecular methods lack the sensitivity and cellular resolution needed to define the role of apoptosis of particular cell types in disease processes. This is especially true for chronic slow degenerative diseases, in which cell death is protracted and asynchronous, and individual apoptotic cells are present for only a limited period of time.

Recombinant DNA technology can be used to alter antibodies, for example, by substituting specific immunoglobulin regions from one species with immunoglobulin regions from another species. Neuberger et al. (PCT publication WO86/01533) describe a process whereby the complementary heavy and light chain variable domains of an immunoglobulin molecule from one species can be combined with the complementary heavy and light chain immunoglobulin constant domains from another species. This process can be employed, for example, to substitute one or more of the constant region domains to create a “chimeric” antibody, which can be employed for human therapy. A chimeric antibody produced as described by Neuberger et al. can have a human Fc region for efficient stimulation of antibody mediated effector functions, such as complement fixation, but still has the potential to elicit an immune response in humans against the “foreign” variable regions.

Winter, British Patent No. GB2188638, describes a process for altering antibodies by substituting the complementarity determining regions (CDRs) with those from another species. This process can be employed, for example, to substitute the CDRs from the murine variable region domains of a monoclonal antibody with desirable binding properties (for instance to a human pathogen) into human heavy and light chain immunoglobulin variable region domains. These altered immunoglobulin variable regions can then be combined with human immunoglobulin constant regions to create antibodies that are totally human in composition except for the substituted murine CDRs. The “reshaped” or “humanized” antibodies described by Winter elicit a considerably reduced immune response in humans compared to chimeric antibodies because of the considerably less murine components. Further, the half life of the altered antibodies in circulation can approach that of natural human antibodies.

Due to the inadequacies of these and other known methods for the detection of cell apoptosis, there continues to be a need for new and selective methods of detecting apoptotic cells. The present invention addresses this and other problems.

III. Antibody Composition of the Present Invention

An antibody composition of the present invention can be employed to detect apoptotic cells, for example those cells in a culture, colony or population undergoing apoptosis. An antibody composition of the present invention, therefore, can be used to discriminate between apoptotic cells and cells that are not undergoing apoptosis. An antibody composition of the present invention specifically recognizes an epitope on the surface of an apoptotic cell, the epitope being detectable in cells undergoing apoptosis and undetectable in cells not undergoing apoptosis.

An antibody composition of the present invention can comprise a fragment of a complete antibody, as well as a complete antibody of any isotype. In one example, an antibody composition takes the form of a dimerized scFv, although an antibody composition of the present invention can take the form of monomeric or multimeric scFv. When the antibody composition is a dimerized scFv, dimerization is can be achieved via leucine zipper elements. Dimerization can be between like elements or between unlike elements as in a heterodimer that can be used in bispecific antigen binding. However, other methods of dimerization can also be employed, such as disufide bond formation between heavy and light chain elements.

The heavy and light chain elements of an scFv can comprise one or more point mutations. Indeed, one advantage of the present invention is the ability to easily and quickly introduce mutations into the scFv. Such mutations can be introduced by employing standard mutagenesis techniques known to those of skill in the art and discussed more fully hereinbelow.

III.A. Variable Heavy (V_{H}) Chain

An scFv of the present invention comprises a variable heavy chain. In one example, a variable heavy chain of an scFv of the present invention comprises a variant of the murine 3H9 antibody. In other embodiments, a heavy chain of an scFv of the present invention comprises one or more mutations from the germline 3H9 sequence. Such mutations can be at any point and can involve any substitution. Preferred mutations, however, comprise the mutations R53G, I57T, D65G, D56R, S76R, N58R, S31R and combinations thereof. A summary of DOPS and β2GPI binding data for several scFv's comprising one or more of the aforementioned (and other) mutations is presented in Table 1 below.

The H chain of 3H9 has been isolated from a hybridoma cell line secreting an IgG2b isotype antibody (Shlomchik et al., (1987) Proc. Natl. Acad. Sci. 84: 9150-9154). The H chain variable (V) gene has been cloned from this hybridoma and used to construct an IgM H chain transgene that has been reinserted into the germline of mice (Erickson et al., (1991) Nature 349: 331-334). These transgenic mice proceeded to synthesize the 3H9 H chain as part of IgM isotype antibodies that, in different B cells and B cell hybridoma lines, were combined with different L chains. In addition, 3H9 and the D56R mutant H chain V genes were used as IgG2b isotype transgenes in mice that secreted these H chains as IgG2b isotype antibodies (Radic et al., (1995) J. Immunol. 155: 3213-3222). Moreover, the 3H9 H chain was used for in vitro mutagenesis and mutant H chains were transfected into hybridoma lines to obtain mutant IgG2b antibodies (Radic et al., (1993) J. Immunol. 150: 4966-4077). More recently, the 3H9 H chain and its variants have been used for the construction and expression of single chain Fv antibodies (Cocca et al., (1999) Prot. Expr. Purif. 17: 290-298). It is known that one polypeptide span that comprises the H chain complementarity determining region 3 (CDR3) can be exchanged between different antibodies and can alter antibody specificity while maintaining binding to phospholipids (Seal et al. (2000) Eur. J. Immunol. 30: 3432-3440). Therefore, the 3H9 H chain can function when it includes mutations that revert its sequence to be more similar to the germline sequence of this V gene, or when it includes several forward mutations, as shown herein. The 3H9 H chain can be expressed with different CDR3 domains and as part of an IgM or IgG antibody molecule and its binding to phospholipid antigens is maintained. Moreover, the 3H9 H chain can be considered as representative of several related V genes from the J558 V gene family that are frequently observed in murine autoantibodies to nucleoprotein and/or phospholipid antigens (Radic and Weigert, (1994) Annu. Rev. Immunol. 12: 487-520). Thus, it is predicted that the binding specificity of 3H9 will be shared with other murine antibodies that express H chains that are structurally and functionally related to the 3H9 H chain.

III.B. Variable Light (V_{L}) Chain

An scFv of the present invention also comprises a variable light chain. In one embodiment, a variable light chain of an scFv of the present invention comprises a variant of the murine 3H9 antibody. In another embodiment, a variable light chain comprises a κ light chain, although other light chains, such as λ light chains, can also be employed in an scFv of the present invention. Preferably, a light chain of an scFv of the present invention comprises one or more mutations from the germline 3H9 sequence. Such mutations can be at any point and can involve any substitution. A summary of DOPS and β2GPI binding data for several scFv's comprising one or more of the aforementioned (and other) mutations is presented in Table 1 below. A variety of experiments have demonstrated that binding to phospholipids and nucleoproteins reflects the immunodominant role of the 3H9 H chain. Thus, a diverse range of L chains can be associated with a 3H9 H chain in its binding to phospholipid or nucleoprotein. This has been demonstrated by chain recombination experiments involving 3H9 H chain transfections of hybridoma cell lines (Radic et al., (1991) J. Immunol. 146: 176-182), as well as by analysis of mice with IgM isotype 3H9 H chain transgenes (Radic et al., (1993) J. Exp. Med. 177: 1165-1173) and IgG isotype 3H9 H chain transgenes (Ibrahim et al., (1995) J. Immunol. 155: 3223-3233). A representative, but non-limiting set of suitable L chains that, in combination with a 3H9 H chain, facilitate phospholipid binding includes the 3H9 L chain itself, encoded by the sequence given in SEQ ID NOs: 6 and 7, the H144 Vk8 L chain encoded by GenBank accession #X17634 (SEQ ID NO: 8), the 84-11 Vk1 L chain encoded by GenBank accession #U29768 (SEQ ID NO: 9), the 84-6 Vk8 L chain encoded by GenBank accession #U29780 (SEQ ID NO: 10), and the 73-17 Vk12/13 L chain encoded by GenBank accession #U30232 (SEQ ID NO: 11), among others.

TABLE 1
Binding of scFv to DOPS or DOPS-β2GPI

| Antibody | DOPS ± SD* | DOPS-β2GPI ± SD* |
|---|---|---|
| 3H9 | 31.33 ± 0.18 | 20.09 ± 0.16† |
| R53G | ND§ | ND§ |
| I57T | 11.78 ± 0.11 | 9.59 ± 0.12 |
| D65G | 12.56 ± 0.15† | 9.08 ± 0.23†‡ |
| R53G/I57T/D65G | 11.72 ± 0.12† | 8.85 ± 0.11†‡ |
| S31R | 9.55 ± 0.08† | 3.44 ± 0.25†‡ |
| D56R | 8.36 ± 0.08† | 3.17 ± 0.26†‡ |
| N58R | 11.83 ± 0.12† | 6.75 ± 0.20†‡ |
| S76R | 10.30 ± 0.14† | 5.89 ± 0.11†‡ |
| D56R/S76R | 5.62 ± 0.22† | 2.01 ± 0.30†‡ |

*Concentrations of scFv (μg/ml) that give 50% maximal binding are listed.
†Significant change from 3H9 (p < .05).
‡Differences in binding between DOPS and DOPS-β2GPI are significant (p < .05).
§Not detected.

III.C. Linker Sequence

A linker sequence can be employed in an scFv of the present invention. Such a linker sequence is disposed, for example, between the V_{H} and V_{L} coding sequences. A function of the linker sequence is to maintain the proper reading frame between the V_{H} and V_{L} coding sequences, thus ensuring that the amino acids comprising the V_{H} and V_{L} sequences are properly expressed and joined.

Suitable linker sequences can be of any length, however it can be desirable that a linker sequence comprise about 15 amino acids, or up to and including about 45 nucleotides. The amino acid composition of the linker sequence can vary. The precise composition of the linker sequence can be tailored to fit a particular desire, such as a desire that the linker sequence comprise a length of hydrophobic or hydrophilic residues. A representative linker sequence comprises the following 15 residues GGGGSSGGGSGGGGS (SEQ ID NO: 1).

III.D. Dimerization Domain

An scFv of the present invention can also comprise one or more dimerization domains. Leucine zippers have been employed to accomplish dimerization in a variety of systems, including the production of bivalent scFv and Fab dimers (Radic & Seal, (1997) Methods 11: 20-26; Pack & Plückthun, (1992) Biochem. 31:1579-1584). Thus, a dimerization domain of an scFv of the present invention can comprise a leucine zipper.

When an scFv of the present invention comprises a leucine zipper, the nucleic acid sequence comprising the leucine zipper can be cloned from any source. The only requirement for a leucine zipper is that it comprises the known leucine zipper motif. In a one example of a leucine zipper motif, a total of at least four leucine residues are spaced seven residues apart (e.g., r, r+7, r+14, r+21).

Leucine zippers can comprise the sequence of the leucine zipper of a variety of proteins, for example c-fos, c-jun, GCN4 (which is a member of the b/zip family) and Max (which is a member of the b/HLH/zip family). In addition, artificial leucine zippers have been constructed by those skilled in the art (Arndt et al., (2001) J. Mol. Biol. 312: 221-228). The c-jun leucine zipper can be a desirable component of an scFv of the present invention due to its ability to form jun-jun homodimers, as well as its ability to form heterodimers with c-fos. On the other hand, while the c-fos leucine zipper can also be employed in the present invention, the c-fos leucine zipper cannot form homodimers and is thus most useful when paired with an scFv comprising a c-jun leucine zipper, with which it can dimerize.

III.E. Histidine Tag

An advantage of an scFv of the present invention is the ease of expression, mutation and purification. These advantages arise, in part, from the ability to express an scFv in a bacterial expression system. The use of a bacterial expression system facilitates purification of an scFv via standard protein purification techniques. However, the purification of an scFv can be further simplified by adding one or more amino acid sequences that can ease purification of an scFv.

One sequence that can be added to an scFv to assist in purification is a hisitidine tag, or “his tag.” A histidine tag generally comprises a plurality of hisitidine residues. Passing the tagged protein over a column comprising a nickel N-(5-amino-1-carboxypentyl)iminodiacetic acid (Ni-NTA) agarose matrix can isolate proteins comprising his tags.

Any number of histidine residues can be added to an scFv to assist in purification. Generally, a sequence of about five histidine residues (i.e. a penta-his tag) is employed, although sequences of more or less histidine residues can be employed. For example, in one embodiment of the present invention, six histidine residues (a hexa-his tag) are employed.

III.F. B Domain of Protein A

An scFv of the present invention can also comprise the B domain of protein A. This sequence can be employed, as a substitute for, or in addition to, a his tag to assist in the purification of an scFv. It is known that the Fc region of human immunoglobulin G (IgG) binds the B domain of protein A. Thus, when an scFv comprises the B domain of protein A, an additional purification strategy is available.

When an scFv comprises both the B domain of protein A and a his tag, a two-step purification process is an option. Thus, purification can be based on both the isolation of an antibody on a Ni-NTA agarose column, and on the interaction of the antibody with the Fc region of human IgG. This two-step purification process has shown to be a significant increase in specific activity over the single step purifications (Cocca et al., (1999) Protein Expres. Purif. 17: 290-298). In one example, the B domain of protein A is the 58 amino acid sequence derived from the Staphylococcus aureus protein A (GenBank Accession No. U54636, version U54636.1, GI: 1480566).

IV. Epitope Properties and Location

In one aspect of the present invention, an epitope recognized by an scFv of the present invention is disposed on the surface of an apoptotic cell. The recognition of an epitope on the surface of an apoptotic cell represents an advance over the prior art. In prior art methods that appear to describe detection of apoptotic cells via antibody binding, the epitope is disposed internally within the cell. For example, U.S. Pat. No. 5,935,801 to Schlossman & Zhang discloses an antibody that appears to bind an epitope on the mitochondrial membrane of apoptotic cells. U.S. Pat. No. 6,048,703 to Siman et al. discloses an antibody that apparently binds to protein fragments generated during apoptosis. The protein fragments forming the epitopes disclosed in Siman et al. are fragments of proteins apparently disposed within the cell that are not accessible on the surface of the cell. Therefore, additional manipulations are required to render these epitopes accessible to antibodies.

An epitope of the present invention, on the other hand, is disposed on the surface of an apoptotic cell. Thus, when employing an antibody of the present invention, e.g., an scFv, there is no need to lyse the cell to determine if it is apoptotic. Nor is it necessary to wait until the cell reaches an overly advanced stage of apoptosis in order to detect the process. Thus, flow cytometry, microscopy and various cell sorting methods can be employed in the present invention to detect apoptotic cells because the epitope recognized by an antibody composition of the present invention is a surface epitope. These and other apoptosis detection methods are described more fully hereinbelow.

An epitope recognized by an antibody composition of the present invention is disposed on the surface of apoptotic cells. Additionally, an epitope can be associated with a bleb structure, which is known to generally accompany the apoptosis process and discussed further hereinbelow. Further, an epitope recognized by an antibody composition of the present invention can be localized to one or more blebs themselves, as well as the regions surrounding the blebs. Thus, an antibody composition of the present invention can recognize an epitope located on a bleb of an apoptotic cell.

V. Expressing an scFv of the Present Invention

A construct can be prepared comprising a polynucleotide sequence encoding an scFv of the present invention. Competent cells (e.g., bacterial cells) can be transformed with the construct, the expression of the scFv can be induced for a determined period of time, and the scFv can subsequently be purified from the cells by employing standard protein purification methods (see, generally, Janson & Rydén (eds), (1998) Protein Purification: Principles, High Resolution Methods, and Applications (2^{nd} ed.), Wiley-Liss, New York, N.Y.). A general procedure for producing an scFv of the present invention follows.

V.A. Engineering a Construct

A construct adapted for expressing an scFv of the present invention can be engineered generally as follows. Nucleotide sequences encoding a variable heavy chain and a variable light chain can be produced by PCR amplification of V_{H} and V_{L} coding regions of a suitable antibody, for example 3H9 (Shlomchik et al., (1987) Proc. Natl. Acad. Sci. U.S.A. 84: 9150-9154; Radic et al., (1993) J. Immunol. 150: 4966-4977). Coding regions for V_{H} and V_{L} can be amplified by employing oligonucleotide primers complementary to one or more codons of the V_{H} and V_{L} chains. In addition, the primers can encode unique restriction endonuclease recognition sites. The V_{H} and V_{L} coding segments are then joined in frame into a single chain Fv by incorporating a synthetic linker peptide. A representative linker sequence comprises a segment encoding the sequence GGGGSSGGGSGGGGS (SEQ ID NO: 1), which is 15 amino acids in length. The coding domains are optimally flanked by restriction sites at the amino terminus of the V_{H} sequence and at the carboxy terminus of the V_{L} sequence. The linker segment can also be set off from the V_{H} and V_{L} coding sequences by introduced restriction sites.

The coding segments can then be cloned into a suitable prokaryotic expression vector. A representative, but non-limiting, list of suitable expression vectors comprises: col E1, pCR1, pBR322, pMB9, pET vectors and their derivatives; wider host range plasmids, such as RP4, phage DNAs, (e.g., the numerous derivatives of phage λ, e.g., NM 989, and other DNA phages, e.g., M13 and filamentous single stranded DNA phages), yeast plasmids and vectors derived from combinations of plasmids and phage DNAs, such as plasmids which have been modified to employ phage DNA or other expression control sequences. The pET26b+ vector, which is available from Novagen, Inc. of Madison, Wis., is an example of one expression vector that can be employed in the present invention. The coding segments can be inserted just downstream of an optional leader sequence, such as the pelB sequence found on the pET26b+ vector, which directs secretion of the recombinant proteins to the periplasmic space. Other expression vectors that can be employed include the members of the pET family of vectors, which are available from Novagen, Inc. of Madison, Wis. A variety of other commercially available vectors can be employed in the present invention.

Additional polypeptide coding regions for leucine zippers can be introduced into the vector at the introduced restriction sites. For example, leucine zippers of murine c-jun, or the murine c-fos (ATCC 41041), which are representative leucine zippers, can be introduced in frame at the 3′ end of the coding region. Both leucine zipper coding regions can be trimmed to 43 codons, the minimum size for efficient dimerization (O'Shea et al., (1989) Science. 245: 646-648) and they can be flanked by polypeptide sequences of variable length at their amino and carboxy termini.

To facilitate rapid and convenient detection and purification of an expressed protein, the coding sequence can also comprise the B domain of a protein A. The 58 amino acid long B domain of the S. aureus protein A is a representative sequence. The protein A sequence is amplified from a suitable clone using suitable primers. Additionally, a his tag can also or alternatively be engineered into the coding sequence, or it can comprise an element of the expression vector. For example, a pentahistidine tag contained in the pET26b+ vector can be accessed by engineering a continued reading frame between the protein A domain and the histidine codons in the vector. Finally, each coding region, or the complete expression vector, can be sequenced using the SEQUENASE™ enzyme and conditions recommended by the manufacturer (U.S. Biochemical Co. of Cleveland, Ohio).

V.B. Expression of an scFv of the Present Invention

An scFv of the present invention can be expressed via the following protocol that finds general application in bacteria. Initially, the cells of an actively growing bacterial culture, (for example, E. coli strain HMS 174 (DE3)), can be transformed with the expression vector via standard transformation techniques. See, e.g., Sambrook et al., (1989) Molecular Cloning: A Laboratory Manual, Cold Spring Harbor Laboratory Press, Cold Spring Harbor, N.Y. The culture is then diluted into a volume of media comprising one or more selection compounds which can assist in selecting transformed cells. In a one embodiment, the medium is 2YT medium and the selection compound is kanamycin.

The culture is then grown at 37° C. with shaking. At an OD₆₀₀ of about 1.0, IPTG (Labscientific, Inc. of Livingston, N.J.) can be added to a concentration of 1 mM and the culture grown with shaking overnight at 22° C. or other suitable temperature. Cells can be harvested by centrifugation and expressed proteins can be recovered from the growth medium by ammonium sulfate precipitation and from the periplasm (if localized to the periplasm) by cell wall digestion and centrifugation.

ScFv's recovered from the bacterial growth medium can be precipitated with ammonium sulfate. The salt (0-75% saturation) is added gradually and dissolved by stirring at 4° C. The pH is adjusted to about 7.6 and the mixture allowed to stand at 4° C. for 1 hr. The precipitated proteins are collected by centrifugation. The pellet is then resuspended in phosphate buffered saline (PBS) or other suitable buffer, and used directly or subjected to further purification.

The periplasmic extract can be obtained by incubating the cell pellet on ice after resuspension in a fraction of the culture volume of a digestion buffer, for example 30 mM Tris-HCl (pH 8.0), 20% sucrose, 1 mM EDTA, 1 mM 4-(2-aminoethyl) benzene-sulfonyl fluoride (AEBSF) (Sigma Chemical Co. of St. Louis, Mo.), and 1 mg/ml lysozyme. Protoplasts are centrifuged and the recovered supernatant used directly or subjected to further purification.

V.C. Purification of an scFv of the Present Invention

An scFv of the present invention can be purified via any of several methods. Many of the purification approaches are facilitated by the selection of the components of a construct. For example, affinity chromatography methods can be employed in a purification protocol and can be facilitated by the choice of a sequence adapted to ease purification of the antibody. For example, inclusion of a his tag can facilitate purification by Ni-NTA chromatography. Alternatively, inclusion of a protein A fragment or domain can facilitate purification by affinity chromatography. IgG-agarose chromatography can generally be employed. These and other purification methods can be employed in the present invention and are described more fully hereinbelow.

V.C.1. Purification of an scFv by Ni-NTA Agarose Chromatography

An scFv comprising a his tag can be purified by employing Ni-NTA chromatography. In a representative embodiment of this scheme, protein samples recovered from the bacterial growth medium and the bacterial periplasm are first dialyzed against Ni-NTA binding buffer (e.g., 20 mM Tris-HCl, pH 8.0; 300 mM NaCl; 10 mM imidazole) overnight at 4° C. An aliquot of dialysate can then be mixed with a volume of 50% Ni-NTA slurry (Qiagen of Valencia, Calif.) that is pre-equilibrated with binding buffer for 2 hours at 4° C. The mixture can then be applied to a poly-prep chromatography column (Bio Rad, of Hercules, Calif.), washed twice with a wash buffer (e.g., 20 mM Tris-HCl, pH 8.0; 300 mM NaCl; 20 mM imidazole; 0.5% Tween 20) and eluted in a volume of an elution buffer (e.g., 20 mM Tris-HCl, pH 8.0; 300 mM NaCl; 250 mM imidazole). The eluate can then be dialyzed against PBS overnight at 4° C.

The purified protein can be stored at 4° C., and can maintain stability with respect to proteolysis and DNA binding for at least 1 month. The dialyzed eluate from the Ni-NTA column can be further purified over IgG agarose following the same procedure as for the purification of the starting protein aliquots.

V.C.2. Purification of an scFv by IgG Agarose Chromatography

Immunoaffinity chromatography can also be employed to purify an antibody of the present invention. In a representative embodiment of this scheme, protein samples from the bacterial growth medium and the bacterial periplasm can be dialyzed against PBS overnight at 4° C. An aliquot of dialysate can then be mixed with a 50% IgG agarose slurry (Jackson Immunoresearch Laboratories of West Grove, Pa.) that is pre-equilibrated with PBS for 2 hours at 4° C. The mixture can then be applied to a chromatography column, washed once with a volume of PBS and eluted sequentially with volumes of 0.1 M glycine (pH 2.7), 1M Tris base (pH 10.7), and 3.5M MgCl₂ in 10 mM NaH₂PO₄ buffer (pH 7.2). The glycine eluate can then be neutralized with a volume of 1.0M Tris-HCl (pH 8.0), while the Tris eluate can be neutralized with a volume of 1.0M NaH₂PO₄ (pH 7.0). All eluates can additionally be dialyzed against PBS overnight at 4° C. and stored at 4° C., which will maintain the stability of the samples for at least about one month.

V.C.3. Protein Purification by Hydroxyapatite

Hydroxyapatite can also be employed as a purification technique, based on the affinity of an scFv of the present invention for this material. In one embodiment of this technique, protein samples recovered from the bacterial growth medium and the bacterial periplasm can be dialyzed against 50 mM NaH₂PO₄ buffer overnight at 4° C. A volume of dialysate is then passed through a hydroxyapatite column at 4° C. The column is then sequentially washed with 50 mM NaH₂PO₄, 0.1 M NaH₂PO₄, 0.2M NaH₂PO₄, and protein is eluted with 0.5M NaH₂PO₄. The eluate is then dialyzed against PBS overnight at 4° C. and stored at 4° C., which will maintain protein stability for at least about one month.

VI. Formation of scFv Dimers

The epitope recognition region of an antibody is generally defined by a three-dimensional cavitous structure. The cavitous structure can be formed by the interlacing or association of structural elements of one or more protein chains with one another. The combining site of one scFv polypeptide can be brought into the proximity of the combining site of another polypeptide with the same or different binding specificity by forming dimers of these polypeptide chains. Thus, it is preferable for an scFv of the present invention to form dimers and thereby define an extended and unique epitope recognition region. Dimerization can be ascertained via the following representative, but non-limiting, embodiment.

A periplasmic extract comprising scFv's, which can comprise a c-jun leucine zipper motif, are heated to 65° C. for 2 minutes and incubated for 30 minutes at room temperature. The ability of c-jun leucine zippers to form homodimers provides for the formation of dimers upon cooling and room temperature incubation.

To assess the extent of dimer formation, the mixture can be applied to a SEPHADEX™ G75 column (available from Amersham Pharmacia Biotech, Inc. of Piscataway, N.J.) or other size exclusion column that has been pre-equilibrated with 50 mM Tris-HCl, pH 8.0, 1 mM EDTA at 4° C. The column is developed with the same buffer and fractions collected. The column can be rinsed with 20 column volumes of buffer between samples. For basis of a comparison, purified bovine serum albumin (BSA), bovine carbonic anhydrase (BCA) or other protein can be run under identical conditions on the same chromatography matrix. The fractions can then be analyzed by SDS-PAGE and/or Western blots.

VII. Formation and Detection of Immunocomplexes

When an antibody composition of the present invention (e.g., scFv or dimerized scFv) recognizes an epitope, the antibody composition associates with the epitope. A “lock-and-key” analogy is often used to describe the interaction between an antibody composition and an epitope: the epitope resembles a key that precisely fits an antibody composition's corresponding structural shape, or “lock,” although electrostatic and conformational considerations must also be taken into account. Non-covalent binding stabilizes the complex and holds it together. An epitope-antibody composition interaction is primarily a result of four forces: van der Waal's forces (dipole-dipole interactions), hydrogen bonds, hydrophobic interactions, and ionic (coulombic) bonding. A range of techniques is available to detect the formation of the antibody composition-epitope immunocomplex.

VII.A. Formation of an Immunocomplex

Formation of an immunocomplex can be easily achieved due to the inherent nature of an antibody composition to associate with its epitope. In a representative, but non-limiting, embodiment, an immunocomplex can be formed as follows. Cells which are to be tested for apoptosis via an scFv of the present invention (e.g., Jurkat cells) are first harvested from culture, resuspended at a density of 1×10⁶ cells/ml in a suitable medium (e.g., RPMI 1640, available from Mediatech, Inc. of Herndon, Va.) comprising 10% fetal bovine serum and grown under a variety of conditions that may induce apoptosis. Following culturing, cells are harvested, aliquoted into tubes and washed with ice-cold Hanks Balanced Salt Solution (Mediatech) comprising 1.0 mM CaCl₂, 3% fetal bovine serum, and 0.02% NaN₃. Washed cells are then incubated with 10 μg/ml of scFv or dimerized scFv on ice and washed twice as above.

VII.B. Detection of an Immunocomplex on the Surface of Intact Cells

An advantage of an antibody composition of the present invention (e.g., an scFv) is its ability to detect an immunocomplex formed on the surface of cells. In this role, the cells are examined in culture; they need not be lysed in order to expose an antigen or epitope. Cells on which an immunocomplex has formed can be detected by a variety of methodologies, including flow cytometry and fixed cell immunofluorescence techniques. A range of cell sorting techniques can also be employed based on the affinity of IgG for an antibody composition of the present invention.

VII.B.1. Flow Cytometry

Flow cytometry is one representative method of detecting an association between an antibody composition of the present invention and an apoptotic cell. Flow cytometry protocols, including those that can be employed in the present invention, typically proceed according to the following general procedure. Cells are ordered into a single row by the fluidic architecture of the flow cytometer instrument. The row of cells is then fed through a light source (laser beams are typically employed), where each cell is irradiated by the beam. The light is scattered by each cell as it is irradiated, which is recorded. The wavelength of the laser can also induce fluorescent emission, which is also recorded. Irradiated cells continue through the fluidic architecture of the instrument and are collected. Often, it is desirable to separate or “gate” cells presenting a predetermined scattering profile, which can be indicative of certain cellular morphologies and/or association with other compounds.

A flow cytometer instrument can measure a range of scattering and fluorescent properties, all of which can be measured simultaneously or sequentially. For example, a flow cytometer typically measures low angle forward scatter intensity, which can provide information on the dimensions of a cell. A flow cytometer can also measure side or orthogonal scatter intensity, which can provide information on intracellular structures. Qualitatively, these two light scattering measurements can provide information on whether a cell is alive or dead and can also be employed to separate cellular and other debris from whole cells. Additionally, fluorescence data can be acquired, which can provide information regarding the association of a label with a cell. Provided that cells are made permeable, fluorescence information can also be indicative of the nucleic acid quantity and/or quality present in a cell.

In one embodiment, cells to be tested for apoptosis are harvested from culture, aliquoted into tubes and washed with ice-cold Hanks Balanced Salt Solution (Mediatech) comprising 1.0 mM CaCl₂, 3% fetal bovine serum, and 0.02% NaN₃. Washed cells are then incubated with 10 μg/ml of scFv or scFv dimer for 15 minutes on ice and washed twice as above, followed by staining with allophycocyanin-conjugated rabbit IgG (Molecular Probes, Eugene, Oreg.) as recommended by the manufacturers. Cells are then analyzed on a flow cytometry system (e.g., the FACSCalibur™ system available from BD Immunocytometry Systems of San Jose, Calif.). In one example, thirty thousand events are collected per sample and analyzed using suitable data analysis software (e.g., the FLOWJO™ software available from Treestar, Inc. of San Carlos, Calif.).

VII.B.2. Confocal Immunofluorescence Microscopy

The formation of an immunocomplex can also be detected via confocal immunofluorescence microscopy, rather than sequentially passing cells through a laser beam, such as the arrangement in a flow cytometer. For example, in one embodiment of an immunofluorescence microscopy arrangement, a secondary antibody (e.g., an anti-scFv antibody) is tagged with a fluorescent moiety and the presence of an immunocomplex is detected by monitoring fluorescence originating with the secondary antibody.

Cells can be prepared for fixed cell immunofluorescence and stained by methods known in the art (see, e.g., Casey et al., (1995) J. Immunol. Meth. 179: 105-116). Briefly, purified (e.g., IgG agarose-purified) scFv is mixed with cells in a test tube at a concentration of about 10 μg/ml in PBS/BSA and incubated for about 30 minutes at room temperature. Unbound scFv can be removed by washing with PBS/BSA and centrifugation. Binding of the scFv to the cells can be visualized by incubation of the fixed cells with fluorescein isothiocyanate-conjugated human or rabbit IgG (FITC-IgG) (Jackson Immunoresearch Laboratories) that, in one embodiment of the present invention, reacts with the B domain of protein A. Unbound fluorochrome can be removed by washing, excess buffer removed by blotting, and cells can be transferred to microscope slides and covered with a small drop of mounting medium (such as GEL/MOUNT™ available from Biomeda, Inc. of Hayward, Calif.) and a coverslip. The fluorescence evaluation can be performed using any fluorescence microscope, for example a LABOPHOT™ microscope available from Nikon. In a one exemplary embodiment, cells can be examined with a Zeiss LSM 510 laser scanning microscope (Carl Zeiss Inc., Thorwood, N.Y.) and the images analyzed with the LSM 510 confocal analysis software.

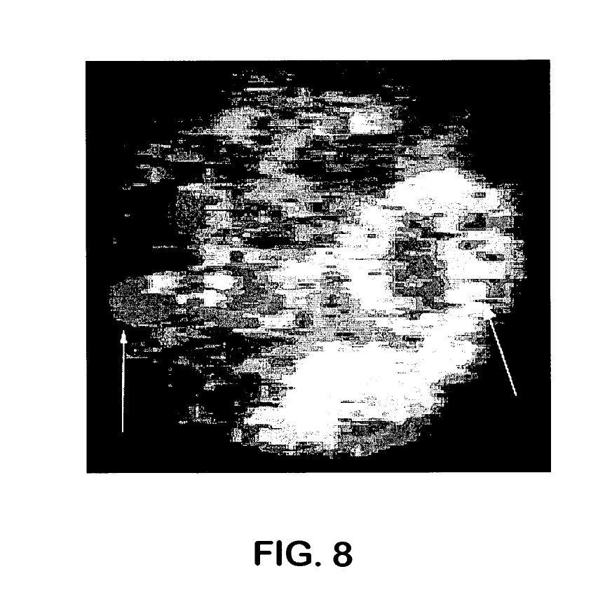

FIG. 8 is a micrograph depicting surface blebs that are recognized by the D56R/S76R scFv and frequently contain fragments of the nucleus. The experiments associated with FIG. 8 are further described in Laboratory Example 9.

VIII. Design, Preparation and Structural Analysis of scFv Polypeptides

The present invention provides for the production of scFv's, including mutant scFv's. Protocols and methods for generating mutations in a sequence comprising SEQ ID NOs: 2, 6 and 7 are provided. In one embodiment, an advantage of the present invention is that since the scFv's are coded on an expression vector and produced in a bacterial system, mutations can be readily introduced into the sequence of an scFv.

VIII.A. Sterically Similar Compounds

In one aspect of the present invention, sterically similar compounds can be formulated to mimic the key portions of an scFv. Such compounds are functional equivalents. The generation of a structural functional equivalent can be achieved by the techniques of modeling and chemical design known to those of skill in the art and described herein. Modeling and chemical design of scFv structural equivalents can be based on the disclosure of the present invention. It will be understood that all such sterically similar constructs fall within the scope of the present invention.

VIII.B. Chimeric scFv Polypeptides

The generation of chimeric scFv polypeptides is also an aspect of the present invention. Such a chimeric polypeptide can comprise an scFv polypeptide or a portion of an scFv that is fused to a candidate polypeptide or a suitable region of the candidate polypeptide. Throughout the present disclosure it is intended that the term “mutant” encompass not only mutants of an scFv polypeptide but chimeric proteins generated using an'scFv as well. It is thus intended that the following discussion of mutant scFv's apply mutatis mutandis to chimeric scFv polypeptides and to structural equivalents thereof.

In accordance with the present invention, a mutation can be directed to a particular site or combination of sites of an scFv. For example, an epitope recognition site can be chosen for mutagenesis. Alternatively, a residue having a location on, at or near the surface of the polypeptide can be replaced, resulting in an altered surface charge of one or more charge units, as compared to an scFv encoded by a nucleic acid sequence comprising SEQ ID NOs: 2, 6 and 7. Alternatively, an amino acid residue in an scFv can be chosen for replacement based on the location in the combining site and its hydrophilic or hydrophobic characteristics.

Such mutants can be characterized by any one of several different properties as compared with an scFv encoded by a nucleic acid sequence comprising SEQ ID NOs: 2, 6 and 7. For example, such mutants can have an altered surface charge of one or more charge units, or can have an increase in overall stability. Other mutants can have altered specificity for an antigen in comparison with, or a higher specific binding activity than, an scFv encoded by a nucleic acid sequence comprising SEQ ID NOs: 2, 6 and 7.

An scFv and/or an scFv mutant of the present invention can be generated in a number of ways. For example, an scFv encoded by a nucleic acid sequence comprising SEQ ID NOs: 2, 6 and 7 can be mutated at those sites identified as desirable for mutation, via oligonucleotide-directed mutagenesis or other conventional methods, such as deletion. Alternatively, mutants of an scFv comprising encoded by a nucleic acid sequence comprising SEQ ID NOs: 2, 6 and 7 can be generated by the site-specific replacement of a particular amino acid with an unnaturally occurring amino acid. In addition, scFv mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

Mutations can be introduced into a DNA sequence coding for an scFv via synthetic oligonucleotides. These oligonucleotides can contain nucleotide sequences flanking the desired mutation sites, a strategy that can be employed in the engineering of an scFv construct. Mutations can be generated in any sequence coding for polypeptide fragments of an scFv.

According to the present invention, an scFv, or a mutated scFv DNA sequence produced by the methods described above or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the polynucleotide sequences surrounding the desired mutant coding sequence, an expression vector can also comprise control sequences encoding a promoter, operator, ribosome binding site, and/or translation initiation signal. Optionally, the expression vector can be regulated further by a repressor gene or various activator genes and usually contains a signal for termination. In some embodiments, where secretion of the polypeptide is desired, nucleotides encoding a “signal sequence” can be inserted prior to an scFv coding sequence. For expression under the direction of the control sequences, a desired polynucleotide sequence must be operatively linked to the control sequences; that is, the sequence must have an appropriate start signal in front of the polynucleotide sequence encoding the polypeptide, and the correct reading frame to permit expression of a sequence under the control of the control sequences and production of the desired product encoded by that sequence must be maintained.

Any of a wide variety of well-known available expression vectors can be employed to express an scFv coding sequence of the present invention. These include for example, vectors comprising segments of chromosomal, non-chromosomal and synthetic DNA sequences, such as various known derivatives of SV40, known bacterial plasmids, e.g., plasmids from E. coli including col E1, pCR1, pBR322, pMB9, pET and their derivatives, wider host range plasmids, e.g., RP4, phage DNAs, e.g., the numerous derivatives of phage λ, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In the various embodiments of the present invention, vectors amenable to expression in a pET-based expression system are employed. The pET expression system is available from Novagen, Inc. of Madison, Wis.

In addition, any of a wide variety of expression control sequences—sequences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage λ, the control regions of fd coat protein, all for E. coli, the promoter for 3-phosphoglycerate kinase or other glycolytic enzymes, the promoters of acid phosphatase, e.g., Pho5, the promoters of the yeast a-mating factors for yeast, and other sequences known to control the expression of genes of prokaryotic or eukaryotic cells or their viruses, and various combinations thereof.

A wide variety of hosts are also useful for producing scFv polypeptides of the present invention, including mutant polypeptides. These hosts include, for example, bacteria, such as E. coli, Bacillus and Streptomyces; fungi, such as yeasts; mammalian cells, such as CHO and COS-1 cells; plant cells; insect cells, such as Sf9 cells; and transgenic host cells. In addition, transgenic animals can be used to express complete IgM or IgG antibodies encoded by the H and L chain V transgenes of 3H9 and its mutants.

It should be understood that not all expression vectors and expression systems function in the same way to express polynucleotide sequences of the present invention, and to produce scFv polypeptides or scFv mutants. Neither do all hosts function equally well with the same expression system. One of skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

In selecting an expression control sequence, a variety of factors should also be considered. These include, for example, the relative strength of the system, its controllability and its compatibility with the polynucleotide sequence encoding an scFv polypeptide of the present invention, with particular consideration paid to the potential for the formation of secondary and tertiary structures and to sequences that render the mRNA message or its polypeptide product susceptible to rapid degradation.

Hosts should be selected by consideration of their compatibility with the chosen vector, the toxicity of a scFv to them, their ability to express mature products, their ability to fold proteins correctly, their fermentation requirements, the ease of purification of a scFv and safety. Within these parameters, one of skill in the art can select various vector/expression control system/host combinations that will produce useful amounts of an scFv. A mutant scFv produced in these systems can be purified by a variety of conventional steps and strategies, including those used to purify an scFv encoded by a nucleic acid sequence comprising SEQ ID NOs: 2, 6 and 7.

Once an scFv mutation(s) has been generated in the desired location, such as an active site or dimerization site, the mutants can be tested for any one of several properties of interest. For example, mutants can be screened for an altered charge at physiological pH. This is determined by measuring the mutant scFv isoelectric point (pl) and comparing the observed value with that of the wild-type parent. Isoelectric point can be measured by gel-electrophoresis according to the method of Wellner (Wellner, (1971) Anal. Chem. 43: 597). A mutant scFv polypeptide containing a replacement amino acid located at the surface of the enzyme, as provided by the structural information of this invention, can lead to an altered surface charge and an altered pl.

VIII.C. Generation of an Engineered scFv Mutant

In another aspect of the present invention, a unique scFv polypeptide mutant can be generated. Such a mutant can facilitate purification and the study of the antigen- and epitope-binding abilities of an scFv polypeptide.

As used in the following discussion, the terms “engineered scFv”, and “scFv mutant” refer to polypeptides having amino acid sequences which contain at least one mutation in the wild-type sequence. The terms also refer to scFv polypeptides which are capable of exerting a biological effect in that they comprise all or a part of the amino acid sequence of an engineered scFv mutant polypeptide of the present invention, or retain all or some or an enhanced degree of the biological activity of the engineered scFv mutant amino acid sequence or protein. Such biological activity can include the recognition of a particular epitope.

The terms “engineered scFv” and “scFv mutant” also includes analogs of an engineered scFv mutant polypeptide. By “analog” is intended that a polynucleotide or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct scFv mutant analogs. There is no need for an engineered scFv mutant polypeptide to comprise all or substantially all of the nucleic acid sequence of SEQ ID NOs: 2, 6 and 7. Shorter or longer sequences are anticipated to be of use in the invention; shorter sequences are herein referred to as “segments”. Thus, the terms “engineered scFv” and “scFv mutant” also includes fusion, chimeric or recombinant engineered scFv mutant polypeptides and proteins comprising sequences of the present invention. Methods of preparing such proteins are disclosed herein above and are known in the art.

VIII.D. Sequence Similarity and Identity

As used herein, the term “substantially similar” means that a particular sequence varies from nucleic acid sequence of SEQ ID NOs: 2, 6 and 7 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retain at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

VIII.D.1. Sequences That are Substantially Identical to an scFv Sequence of the Present Invention

Nucleic acids that are substantially identical to a nucleic acid sequence encoding an scFv of the present invention, e.g., allelic variants, genetically altered versions of the gene, etc., bind to an scFv-encoding sequence under stringent hybridization conditions. By using probes, particularly labeled probes of DNA sequences, one can isolate homologous or related genes. The source of homologous genes can be any species, e.g., primate species; rodents, such as rats and mice, canines, felines, bovines and equines to name just a few.

Between mammalian species, e.g., human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence will usually be at least about 12 nucleotides long, more usually at least about 30 nucleotides long, and can extend to the complete sequence that is being compared. Algorithms for sequence analysis are known in the art, such as BLAST, described in Altschul et al., (1990) J. Mol. Biol. 215: 403-10. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, (1989) Proc. Natl. Acad. Sci. U.S.A. 89:10915.

In addition to calculating percent sequence identity, the BLAST algorithm also performs a statistical analysis of the similarity between two sequences. See, e.g., Karlin & Altschul, (1993) Proc. Natl. Acad. Sci. U.S.A. 90: 5873-5887. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Percent identity or percent similarity of a DNA or peptide sequence can also be determined, for example, by comparing sequence information using the GAP computer program, available from the University of Wisconsin Geneticist Computer Group. The GAP program utilizes the alignment method of Needleman & Wunch, (1970) J. Mol. Biol. 48: 443-453, as revised by Smith & Waterman, (1981) Adv. Appl. Math. 2: 482-489. Briefly, the GAP program defines similarity as the number of aligned symbols (i.e., nucleotides or amino acids) which are similar, divided by the total number of symbols in the shorter of the two sequences. The preferred parameters for the GAP program are the default parameters, which do not impose a penalty for end gaps. See, e.g., Schwartz & Dayhoff, (1978) in Atlas of Protein Sequence and Structure (Dayhoff, ed.) Washington, D.C.: National Biomedical Research Foundation, 5: 353-358, and Gribskov & Burgess, (1986) Nucl. Acids. Res. 14: 6745-6763.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g., TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs: 2, 6 and 7; or (b) the DNA analog sequence is capable of hybridization with DNA sequences of (a) under stringent conditions and which encode a biologically active scFv gene product; or (c) the DNA sequences are degenerate as a result of alternative genetic code to the DNA analog sequences defined in (a) and/or (b). Substantially identical analog proteins and nucleic acids will have between about 70% and 80%, preferably between about 81% to about 90% or even more preferably between about 91% and 99% sequence identity with the corresponding sequence of the native protein or nucleic acid or part of an antibody composition of the present invention. Sequences having lesser degrees of identity but comparable biological activity are considered to be equivalents.

As used herein, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, representative conditions are salt concentration of about 200 mM and temperature of about 45° C. One example of such stringent conditions is hybridization at 4×SSC, at 65° C., followed by a washing in 0.1×SSC at 65° C. for one hour. Another exemplary stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

As used herein, the term “complementary sequences” means nucleic acid sequences that are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions are specified above.

VIII.D.2. Functional Equivalents of an scFv Nucleic Acid Sequence of the Present Invention

As used herein, the term “functionally equivalent codon” is used to refer to codons that encode the same amino acid, such as the AGC and AGU codons for serine. An scFv-encoding nucleic acid sequence comprising SEQ ID NOs: 2, 6 and 7 which has functionally equivalent codons are covered by the present invention. Thus, when referring to the sequence example presented in SEQ ID NOs: 2, 6 and 7 applicants contemplate substitution of functionally equivalent codons into the sequence example of SEQ ID NOs: 2, 6 and 7. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N-or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within immunoglobulin and other genes.

VIII.D.3. Biological Equivalents

The present invention envisions and includes biological equivalents of scFv polypeptide of the present invention. The term “biological equivalent” refers to proteins having amino acid sequences which are substantially identical to the amino acid sequence of an scFv of the present invention and which are capable of exerting a biological effect in that they are capable of recognizing an epitope present on the surface of an apoptotic cell.

For example, certain amino acids can be substituted for other amino acids in a protein structure without appreciable loss of activity or interactive capacity. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of an scFv polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence example presented in SEQ ID NOs: 2, 6 and 7, applicants envision substitution of codons that encode biologically equivalent amino acids, as described herein, into the sequence example of SEQ ID NOs: 2, 6 and 7. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g., substitution of lie for Leu. Changes designed by man can be introduced through the application of site-directed mutagenesis techniques, e.g., to introduce improvements to the antigenicity of the protein or to test an scFv polypeptide of the present invention in order to modulate epitope recognition or other ability, at the, molecular level.

Amino acid substitutions, such as those which might be employed in modifying an scFv polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of skill in the art. It is implicit in the above discussion, however, that one of skill in the art can appreciate that a radical, rather than a conservative substitution is warranted in a given situation. Non-conservative substitutions in scFv polypeptides of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within ±2 of the original value is preferred, those which are within ±1 of the original value are particularly preferred, and those within ±0.5 of the original value are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 of the original value is preferred, those which are within ±1 of the original value are particularly preferred, and those within ±0.5 of the original value are even more particularly preferred.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 6 and 7, nor is it limited to the vector sequence of SEQ ID NO: 2. Recombinant vectors and isolated DNA segments can therefore variously include an scFv polypeptide-encoding region itself, include coding regions bearing selected alterations or modifications in the basic coding region, or include larger polypeptides which nevertheless comprise an scFv polypeptide-encoding region or can encode biologically functional equivalent proteins or polypeptides which have variant amino acid sequences.

Biological activity of an scFv polypeptide can be determined, for example, by scFv binding assays known to those of skill in the art and disclosed herein. For example, biologically functional equivalent proteins can be identified by expression of scFv on the surface of filamentous phage and screening for binding to apoptotic cells by using flow cytometry, as detailed in U.S. Pat. No. 6,265,150. Further, constructs of 3H9 can be formed in the phagemid pCK13 for expression as fusion proteins to the surface protein encoded by gene III of M13 (Seal et al., (2000) Eur. J. Immunol. 30: 3432-3440). In this way, substantially divergent scFv can be identified, even if they are derived from germlne genes different from 3H9 and even if they are derived from species other than mouse.)

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length preferably being limited by the ease of preparation and use in the intended recombinant DNA protocol. For example, nucleic acid fragments can be prepared which include a short stretch complementary to a nucleic acid sequence set forth in SEQ ID NOs: 6 and 7, such as about 10 nucleotides, and which are up to 10,000 or 5,000 base pairs in length. DNA segments with total lengths of about 4,000, 3,000, 2,000, 1,000, 500, 200, 100, and about 50 base pairs in length are also useful.

The DNA segments of the present invention encompass biologically functional equivalents of scFv polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques. Various site-directed mutagenesis techniques are known to those of skill in the art and can be employed in the present invention.

The invention further encompasses fusion proteins and peptides wherein an scFv coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a gene encoding an scFv component (e.g., a heavy or light chain of a 3H9 antibody), as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages will be gained by positioning the coding DNA segment under the control of a recombinant, or heterologous, promoter. As used herein, a recombinant or heterologous promoter is a promoter that is not normally associated with a gene encoding an scFv component in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (see, e.g., Sambrook and Russell., (2001) Molecular Cloning: A Laboratory Manual, Third Edition, Cold Spring Harbor Laboratory Press, Cold Spring Harbor, N.Y., specifically incorporated herein by reference). The promoters employed can be constitutive or inducible and can be used under the appropriate conditions to direct high level expression of the introduced DNA segment, such as is advantageous in the large-scale production of recombinant proteins or peptides. One representative promoter system contemplated for use in high-level expression is a T7 promoter-based system.

IIIV.E. Summary of Mutations and scFV Elements

Table 2 comprises the sequence of a scFV of the present invention. The various mutations introduced into the DNA sequence are highlighted. The noted mutations include the incorporation of cut sites, as well as point mutations. Some of the structural features of the constructs are indicated. Each cut site, mutation, etc. introduced into scFV sequence is referred to herein by an individual SEQ ID NO; these SEQ ID NOs are disclosed herein and are not provided in Table 2. Table 2 encompasses each of the sequences disclosed in the various aspects of the present invention and represents a cumulative depiction of the sequences noted herein and in the Sequence Listing.

TABLE 2
Summary of Introduced Cut Sites, Mutations and scFV Elements

Met
1 ccatggttcaac tgcagcagtc cggacctgag ctggtgaagc ctggggcctc agtgaagatt

| | |
|---|---|
| | (NcoI)−>VH (BspEI) |
| 61 | tcctgcaagg tttctggcta tgcattcagt agctcctgga tgaactgggt gaagcagagg |
| | aggtcc (S31R) |
| 121 | cctggaaaagg gtcttgagtg gattggacgg atttatccta gagatggaga tattaattac |
| | cctgga (R53G) |
| | ggacgtatt (DS6R) |
| | ga tactaat (157T) |
| | attcgt (NS8R) |
| 181 | aatgggaagt tcaaggacaa ggccacactg actgcagaca aatcctccag cacagcctac |
| | t tcagg (K64R) ag aaca (S76R) |
| | aagggcaa (D65G) |
| 241 | atgcaactca gcagcctgac atctgaggac tctgcggtct acttctgtgc aagagcgagg |
| gcgggg (R96G) | |
| 301 | agtaaatatt cctatgttat ggactactgg ggtcaaggga cctcagtcac cgtgagctcc |
| 361 | ggtggaggag gttcctcagg tggtggatcg ggtcggggtg gatccgaaaa tgtgctgacc |
| | Linker (BamHI)−>VL |
| 421 | cagtctccag caatcatggc cgcatctcca ggggagaagg tcaccatcac ctgcagtgcc |
| 481 | agctcaagtg taagttctgg taactttcac tggtatcagg agaagccagg ctcttctccc |
| 541 | aaactctgga tttataggac atctaacctg gcttctggag tccccgctcg cttcagtggc |
| 601 | agtgggtctg ggacctctta ctctcttaca atcagcagca tggaggccga agatgctgcc |
| 661 | acttattact gccagcagtg gtgtggttac ccattcacgt tcggcacggg gacaaaattg |
| 721 | gagatctcgg gaggtggaaa cgctcggcta gaggaaaaag tgaaaacctt gaaagcgcaa |
| | (BglII)−> c-Jun Leucine Zipper |
| 781 | aactccgagc tggcatccac ggccaacatg ctcagggaac aggtggcaca gcttaagcag |
| 841 | aaagtcatga accactcgagtgatccgaaa gctgacaaca aattcaacaa agaacaacaa |
| | (XhoI) −> Protein A tag |
| 901 | aatgctttct atgaaatctt acatttacct aacttaaatg aagaacaacg caatggtttc |
| 961 | atccagtctc tgaaagatga tccaagccaa agcgctaacc ttttagcaga agctaaaaag |
| 1021 | ctaaatgatg cacaagcacc aaaagctagc ttgcggccgc |
| (NotI) | |

IX. Labels Useful for Identifying Apoptotic Cells

A variety of compounds can be useful for identifying apoptosis-related phenomena and can be employed alone or as part of a double screening protocol. Several compounds that can be employed in the present invention include Annexin V, propidium iodide and IgG conjugates. These and other compounds are discussed in the following sections.

IX.A. Annexin V

Annexins are a family of proteins having anticoagulant properties and, notably, calcium dependent phospholipid-binding ability. Annexin V can specifically measure, or be adapted to specifically measure, apoptotic events, based on its calcium dependent phospholipid binding ability. Notably, Annexin V binds phosphatidylserine in a calcium dependent manner. Phosphatidylserine is a phospholipid bearing a negative charge and is normally disposed on the inner surface of the plasma membrane of a cell. The association of Annexin V with phosphatidylserine can be employed as a identifier of apoptotic cells due to the fact that this lipid is primarily found on the inner surface of a cell membrane, but is translocated to the outer surface upon cell death. Thus, the presence of phosphatidylserine on the outer membrane surface can serve as an indicator of apoptotic activity. Generally, apoptotic cells become sensitized to Annexin V staining after nuclear condensation has begun, but prior to the time when the cell becomes permeable to other molecules. When employed alone, or in conjunction with a viability stain, Annexin V can be employed to identify cells undergoing apoptotic mediated cell death.

The presence or absence of accessible phosphatidylserine (and therefore apoptotic cells) can be determined, for example, by exposing cells to Annexin V that has been tagged with a detectable label, for example FITC-labeled Annexin V, followed by removal of unbound label by filtration. In one embodiment, sample wells having bottoms comprising membrane filters can be employed in the Annexin V staining process, since filtration and subsequent analysis can occur without the need for sample transfer or additional processing steps.

IX.B. Propidium Iodide

Propidium iodide (PI) staining can also be employed in the present invention to identify cells undergoing necrosis or those that are in the late stages of apoptosis. Propidium iodide is a stain that intercalates with chains of nucleic acids, such as DNA and can be a source of red fluorescence upon intercalation. As is known in the art, propidium iodide is specifically intercalated by double-stranded nucleic acids. Since PI can diffuse into the nucleus of dead (necrotic) or late stage apoptotic cells, but cannot penetrate the membrane of viable cells; PI is only a marker of late stage apoptosis. This is, in part, because as apoptosis proceeds, the cell membrane becomes permeable to certain molecules, such as PI.

IX.C. Labeled Immunoglobulin G

Labeled immunoglobulin G (IgG) can be employed to detect association of an antibody composition of the present invention with an epitope on the surface of an apoptotic cell. For example, human or rabbit IgG will recognize the protein A moiety of an scFv of the present invention. Thus, a human or rabbit IgG can be conjugated with a detectable label which, when exposed to an scFv of the present invention, can identify the presence of the scFv. In one representative embodiment, the IgG is isolated from rabbit serum and labeled with allophycocyanin. Labeled IgG can take a role in scFv identification in a variety of techniques, such as flow cytometry and confocal microscopy.

When labeled IgG is employed in flow cytometry-based detection methods, the following exemplary embodiment can be employed. First, cells are washed in a suitable medium, such as Hank's Balanced Salt Solution and then incubated with the scFV. After removing unbound scFv by washing, allophycocyanin-conjugated rabbit IgG can then be added, unbound IgG removed and staining profiles acquired by flow cytometry, followed by analysis of the staining profiles by suitable software. Additional stains can also be employed at the discretion of the researcher.

When labeled IgG is employed in microscopy-based detection methods, the following representative embodiment can be employed. Cells are first washed with a suitable buffer and fixed with ice-cold 4% paraformaldehyde. Fixed cells are then washed again with buffer and incubated with an scFv on ice. After incubation with the scFv, unbound scFv is washed away and bound scFv is detected with a fluorescently-labeled IgG (e.g., Rhodamine Red-conjugated human serum IgG). Additional stains can also be employed at the discretion of the researcher. Following staining cells are again washed with buffer and mounted onto poly-L-lysine-coated glass slides for viewing with a suitable microscope (e.g., a laser scanning microscope).

IX.D. Labeled Antibody Compositions

An antibody composition of the present invention can also be employed to directly detect the presence of an apoptotic cell. This can be achieved via contacting a labeled antibody composition of the present invention with a cell known or suspected of being apoptotic. The labels most commonly employed for these studies are radioactive elements, enzymes, chemicals that fluoresce when exposed to ultraviolet light, chemiluminescent compounds, bioluminescent compounds and others. The label can be subsequently detected via spectroscopic, radiologic and/or other suitable techniques.

Any of the wide range of available fluorescent labels can be employed to detectably label an antibody composition of the present invention. These include, for example, fluorescein, rhodamine, auramine, Texas Red, AMCA blue, Oregon green and Lucifer Yellow. In one embodiment, an isothiocyanate can be employed as a bridging agent to associate the label with an scFv of the present invention. When activated by illumination with light of a particular wavelength, a fluorochromelabelled moiety adsorbs the light energy, inducing a state to excitability in the molecule, followed by emission of the light at a characteristic color that is visually detectable with a light microscope. A consideration when selecting a fluorescent label is the wavelengths at which the label absorbs and emits energy.

An antibody composition can also be labeled with a detectable radioactive element. The radioactive label can be detected by any available counting procedure. Those of skill in the art will recognize the suitability of a given isotope for labeling an scFv, however, representative isotopes comprise ³H, ¹⁴C, ³²P, ³⁵S, ³⁶Cl, ⁵¹Cr, ⁵⁷Co, ⁵⁸Co, ⁵⁹Fe, ⁹⁰Y, 125I, ¹³¹I, ¹⁸⁶Re and ⁹⁹Tc (technetium, used in tumor imaging with scFv).

An antibody composition can also be labeled with an enzyme. Enzyme labels are likewise useful, and can be detected by any of the presently utilized calorimetric, spectrophotometric, fluorospectrophotometric, amperometric or gasometric techniques. Generally, the enzyme is conjugated to a selected particle (e.g., an scFv of the present invention) by reaction with bridging molecules such as carbodiimides, diisocyanates, glutaraldehyde and the like. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine, 5-aminosalicyclic acid, or toluidine is commonly used. It is also possible to employ a fluorogenic substrate, which yields a fluorescent product rather than the chromogenic substrates noted above. In all cases, the enzyme-labeled antibody composition is added to a test sample (e.g., an apoptotic cell), allowed to bind, and then the excess reagent is washed away. A solution containing the appropriate substrate is then added to the formed complex. The substrate will react with the enzyme linked to the structure (e.g., the enzyme conjugated to an scFv), giving a qualitative visual signal, which can be further quantitated, usually spectrophotometrically, to give an indication of a degree of binding which occurred. Many enzymes that can be used in these procedures are known and can be utilized to facilitate the detection of a labeled antibody composition of the present invention. A representative, but non-limiting, list of enzymes that can be employed in the present invention comprises peroxidase, β-glucuronidase, β-D-glucosidase, β-D-galactosidase, urease, glucose oxidase plus peroxidase and alkaline phosphatase. Additionally, U.S. Pat. No. 4,016,043 is referred to by way of example for their disclosure of alternate labeling material and methods.

X. Preparation of Control Samples

When preparing mutants, chimeras and other variants of the scFv encoded by a nucleic acid sequence comprising SEQ ID NOs: 2, 6 and 7, all of which are aspects of the present invention, it can be desirable to assess a degree to which these scFv's recognize apoptotic cells. Thus, when screening and preparing such scFv's a system of control samples can be advantageously applied. A representative, but non-limiting, system of controls is disclosed hereinbelow.

X.A. Induction of Apoptosis

Staurosporine is known to cause the rapid death by apoptosis of a number of cell types (e.g., Jacobson et al., (1993) Nature 361: 365-369; Falcieri et al., (1993) Biochem. Biophys. Res. Commun. 193: 19 (1993); Bertrand et al., (1993) Exp. Cell Res. 207: 388-97). Other compounds are known to have a similar effect, such as camptothecin or anti-Fas antibodies. In a preferred embodiment, Jurkat cells can be harvested from culture and resuspended in a suitable buffer (e.g., RPMI 1640 containing 10% FBS). Apoptosis can then be induced for 12 hours with 1.0 μM staurosporine, 2.0 μM camptothecin (Sigma), or 2.0 μg/ml anti-Fas antibody.

X.B. Inhibition of Apoptosis

It can also be advantageous to prepare cultures in which apoptosis is inhibited. These cultures can provide a baseline against cultures in which apoptosis is occurring. In one embodiment, cultures parallel to those in which apoptosis was induced are pre-incubated with 20 μM z-Val-Ala-Asp(Ome)-fluoromethylketone (z-VAD(Ome)-FMK). To inhibit membrane blebbing, parallel cultures are pre-incubated for with 10 μM Y-27632 (available from Tocris of Ballwin, Mo.). At the end of the incubation period, cells are aliquoted into tubes and stained for flow cytometry or confocal microscopy as described above.

XI. Applications of the Present Invention

There are many applications for the present invention. Of primary, but not exclusive, interest is the detection of apoptotic cells via the disclosed antibody compositions. Other applications for the present invention include the evaluation of the efficacy of a candidate therapeutic compound adapted to effect a change in apoptosis, detecting modulation of apoptosis and providing a kit for the detection of apoptotic cells. These applications of the present invention are discussed hereinbelow; however, it is noted that these are only representative applications of the present invention. Additional applications will be apparent to those of skill in the art upon consideration of the present disclosure.

XI.A. Method of Detecting Apoptotic Cells

The present invention can be employed to detect apoptotic cells. In a representative embodiment, the method comprises contacting an antibody composition adapted to recognize an epitope on the surface of an apoptotic cell with a cell and detecting association of the antibody composition with the cell, the association being indicative of the presence of an apoptotic cell. An interaction between the antibody composition and the cell can be detected via a range of detection strategies and techniques, some of which are disclosed hereinabove. In this embodiment, an epitope can be expressed on the surface of apoptotic cells; however, the epitope is not expressed on the surface of cells that are not undergoing apoptosis.

An antibody composition of the present invention can be generated by employing the methods disclosed hereinabove. In one embodiment, the antibody composition comprises an scFv, which comprises a variable heavy (V_{H}) chain, a variable light (V_{L}) chain, a linker sequence, a dimerization domain and an optional purification tag, such as a his tag or the B domain of a protein A. An scFv comprising these elements can be generated by expressing a construct comprising a nucleic acid sequence encoding the scFv in a suitable expression system (e.g., a bacterial expression system). The construct can be prepared using standard cloning methodology. Purification of the scFv can be achieved via the engineered purification aid alone or in combination with additional protein purification methods.

The cell can be a cell capable of undergoing apoptosis. The antibody composition can be contacted with a cell under conditions suitable to maintain the integrity of the cell. For example, a cell can be maintained in supplemented Hanks Balanced Salt Solution (available commercially from Mediatech of Herndon, Va.) comprising 1.0 mM CaCl₂, 3% FBS, and 0.02% NaN₃. The cell can then be incubated with one or more scFv's for 15 minutes on ice and unbound scFv removed by washing twice with a suitable medium (e.g., HBSS).

Following contacting the antibody composition with the cell, binding of the antibody composition with the cell can be detected. Detection of the formation of such an immunocomplex can be achieved by any of the detection methods disclosed hereinabove, or another detection method known to those of skill in the art. Representative methods of detecting the formation of an immunocomplex include flow cytometry and fluorescence microscopy; however, any detection method can be employed. The choice of detection method is determined, in part, by the selection of a label or reporter. Thus, if a radiolabel is associated with an antibody composition or a cell, an appropriate detection method relies on this activity of this label.

An antibody composition of the present invention is adapted to recognize an epitope present on the surface of an apoptotic cell. This epitope is not present on the surface of non-apoptotic cells. Therefore, recognition of the epitope by an antibody composition of the present invention is indicative that the cell is undergoing apoptosis. Thus, if an antibody composition is detected and associated with a cell, the cell is undergoing apoptosis.

XI.B. Method Of Evaluating The Efficacy of a Candidate Therapeutic Compound Adapted to Effect a Change in Apoptosis

The present invention also provides a method of evaluating the efficacy of a candidate therapeutic compound adapted to effect a change in apoptosis. In a representative, but non-limiting, embodiment of the method, the method comprises contacting an antibody composition adapted to recognize an epitope on the surface of an apoptotic cell with a first sample comprising cells capable of apoptosis; quantifying an extent to which apoptosis is occurring in the first sample; contacting a candidate therapeutic with a second sample comprising cells capable of apoptosis; contacting the antibody composition with the cells of the second sample; determining a second degree to which apoptosis is occurring; and comparing the first and second degrees of apoptosis.

An antibody composition can be engineered and purified by employing the methods disclosed herein. The first sample can comprise any cells, however many embodiments the cells are adapted to undergo apoptosis. The contacting of the antibody composition with the sample can be achieved as described herein.

The step of quantifying an extent to which apoptosis is occurring can be performed by employing an immunocomplex detection scheme that facilitates a quantitative assessment of antibody composition binding. Such a system can be based, for example, on fluorescence, absorbance or radioemission. Although many systems can provide qualitative information regarding antibody composition binding, it is a requirement of this method that a quantitative assessment of antibody composition binding be performed. Automated systems such as flow cytometry instruments can easily perform the quantitative analysis.

Next, a candidate therapeutic is contacted with a second sample. In one example, the second sample can be a parallel culture with the first sample. In this embodiment, both the first and second samples originate from the same source culture. This is advantageous because it provides a baseline against which a degree of apoptosis can be gauged.

The antibody composition contacted with the first sample is then contacted with the second sample. The antibody composition can be contacted with the second sample while the second sample is in the presence of the candidate therapeutic; alternatively, the candidate therapeutic can be removed before the antibody composition is contacted with the second sample. Again, conditions of this second contacting can be the same as were those under which the first contacting was performed. By maintaining the same contacting conditions (e.g., the same medium composition, same temperatures, etc), artifactual and/or misleading data is minimized.

Subsequently, a degree of apoptosis present in a second sample is quantified. For the abovementioned reasons, it can be desirable to quantify the second degree via the same instrumentation and data analysis protocols employed in the generation of the first degree.

The first and second degrees of apoptosis are then compared to gauge the effect of the candidate therapeutic on apoptosis. The comparison can be a direct comparison between the two samples, if the same conditions were employed for each of the contactings. The comparison can comprise a statistical analysis of the data. If the comparison indicates that the second degree is significantly less than the first degree, it can be inferred that the candidate therapeutic compound inhibited or impeded the process of apoptosis. Conversely, if the second degree is significantly greater than the first degree, it can be inferred that the candidate therapeutic promoted the apoptotic process. If the degrees are approximately equal, or there is no significant difference between the two degrees, it can be inferred that the candidate therapeutic did not have a significant effect on the apoptotic process.

By employing this method, a candidate therapeutic can be screened for its ability to effect a change in the apoptotic process of a cell. The method can be quickly and easily performed, thereby making it possible to screen many compounds. Additionally, it is possible to automate the method, thereby removing the need for a researcher to oversee and carry out the method.

XI.C. Kit for Detecting Apoptotic Cells

A kit for detecting apoptotic cells is an aspect of the present invention. In one embodiment, the kit comprises an antibody composition that specifically recognizes an epitope on the surface of an apoptotic cell; a cell culture medium and a detection mechanism adapted to indicate the formation of an immunocomplex between the antibody or antibody fragment and an epitope on apoptotic cell(s).

In some embodiments, the kit comprises an scFv of the present invention. An scFv(s) can comprise, for example, an scFv having the amino acid sequence encoded by a nucleic acid sequence comprising SEQ ID NOs: 2, 6 and 7. Such an scFv is adapted to recognize an epitope on the surface of an apoptotic cell. Additionally, an scFv, by virtue its ability to recognize an epitope on the surface of an apoptotic cell, can differentiate between apoptotic and viable cells.

A cell culture medium comprises an element of the kit. The medium can be employed to maintain cells to be tested for the presence of apoptosis, and can also operate to provide a suitable buffered medium in which a test reaction can be performed.

The kit also comprises a detection mechanism adapted to indicate the formation of an immunocomplex between an antibody composition and an epitope. The detection mechanism can be adapted to directly or indirectly detect the formation of an immunocomplex. In direct detection, an antibody composition can be tagged with a detectable label. The detectable label can comprise a radioisotope, a fluorescent moiety or any other structure that can be directly detected, for example, via absorption or emission spectroscopy. FACS, for example, can be employed to detect an immunocomplex comprising a direct detection label.

When an indirect detection mechanism is employed, the detection mechanism can comprise a secondary label. For example, a labeled anti-scFv IgG can be employed to detect the presence of an immunocomplex. Such an IgG can be conjugated with a detectable label and can be incubated in the presence of a formed immunocomplex. The detectable label can comprise a radioisotope, a fluorescent moiety or any other structure that can be directly detected, for example, via absorption or emission spectroscopy. Alternatively, an enzyme can be conjugated to an indirect detection element a byproduct of a reaction catalyzed by the employed, generally following the conceptual framework of an ELISA assay. Again, FACS can be employed to detect an immunocomplex comprising an indirect detection label.

The general steps of employing a kit for detecting apoptotic cells, then, comprise disposing cells to be tested in the culture medium, incubating the scFv with the cells, and detecting an immunocomplex formed between the scFv and an epitope on the surface of an apoptotic cell.

XI.D. Method of Screening Antibodies

In yet another aspect of the present invention, a method of screening a population of antibodies to identify an antibody adapted to detect cells undergoing apoptosis is disclosed. Antibodies identified as those adapted to detect cells undergoing apoptosis can then be employed in assays designed to identify apoptotic cells. In one embodiment, the method comprises providing a library comprising one of a population of diverse antibodies and a phage display library comprising an antibody fusion protein to be screened. As used herein, the term “population of diverse antibodies” means a plurality of antibodies having different variable regions. Phage display libraries can be constructed using techniques known in the art and described herein.

Next, the library is contacted with a population of cells comprising apoptotic cells to thereby form a mixture. The contacting can be performed by, for example, washing a culture comprising the population of cells over the library. The population of apoptotic cells can be grown under conditions known to those of skill in the art to be conducive to cell growth and/or apoptosis.

Subsequently, the mixture is contacted with a 3H9-derived antibody composition adapted to specifically recognize an epitope on the surface of an apoptotic cell, the epitope being detectable in cells undergoing apoptosis and undetectable in cells not undergoing apoptosis to thereby form a detection mixture comprising bound antibodies. A suitable 3H9-derived antibody composition can be prepared as described herein.

The detection mixture is then contacted with a detectably labeled antibody adapted to recognize the 3H9-derived antibody composition, thereby identifying the presence of apoptotic cells. As described herein, a detectably labeled antibody can comprise an antibody and any detectable label. Representative detectable labels include fluorescent labels and radioactive labels. Techniques for detectably labeling proteins, including antibodies, are known in the art and can be employed in the present invention.

Continuing, apoptotic cells are then separated from non-apoptotic cells. The separation can be achieved by employing suitable columns or plates. For example, an antibody-mediated temporary immobilization of apoptotic cells can be advantageously employed and non-immobilized cells can be washed away from the immobilized apoptotic cells. In some situations, another technique for separating cells that can be employed is FACS.

After separating apoptotic cells from non-apoptotic cells, any bound antibodies are then eluted. Elution can be performed, for example, by employing a buffer adapted to disrupt any interactions between the antibodies and any structure with which the antibodies are associated. A suitable buffer can be, for example, a buffered salt wash.

XII. Generation of Antibodies

In another embodiment of the present invention, antibodies can be derived from a spontaneously autoimmune animal, for example a mouse, as described herein.

In still another embodiment, the present invention provides an antibody composition immunoreactive with an epitope of the present invention. In one embodiment, an antibody composition of the invention is a monoclonal antibody. Techniques for preparing and characterizing antibodies are well known in the art (see, e.g., Howell & Lane, Antibodies A Laboratory Manual, Cold Spring Harbor Laboratory, 1988).

Briefly, a polyclonal antibody composition is prepared by immunizing an animal with an immunogen comprising an epitope of the present invention, and collecting antisera from that immunized animal. A wide range of animal species can be used for the production of antisera. Because of the relatively large blood volume of rabbits, a rabbit represents one animal that can be employed in the production of polyclonal antibodies.

As is well known in the art, a given immunogen can vary in its immunogenicity. It is often necessary therefore to couple the immunogen of with a carrier. Exemplary carriers are keyhole limpet hemocyanin (KLH) and bovine serum albumin (BSA). Other albumins such as ovalbumin, mouse serum albumin or rabbit serum albumin can also be used as carriers.

As is also well known in the art, immunogenicity to a particular immunogen can be enhanced by employing non-specific stimulators of the immune response known as adjuvants. Exemplary adjuvants include complete Freund's adjuvant, incomplete Freund's adjuvants and aluminum hydroxide adjuvant.

The amount of immunogen employed in the production of polyclonal antibodies varies, inter alia, upon the nature of the immunogen as well as the animal used for immunization. A variety of routes can be employed to administer the immunogen, e.g. subcutaneous, intramuscular, intradermal, intravenous and intraperitoneal. The production of polyclonal antibodies can be monitored by sampling blood of the immunized animal at various points following immunization. When a desired level of immunogenicity is obtained, the immunized animal can be bled and the serum isolated and stored.

A monoclonal antibody of the present invention can be readily prepared through use of well-known techniques such as the hybridoma techniques exemplified in U.S. Pat. No 4,196,265 and the phage-display techniques disclosed in U.S. Pat. No. 5,260,203, the contents of which are herein incorporated by reference.

A typical technique involves first immunizing a suitable animal with a selected antigen (e.g., an epitope of the present invention) in a manner sufficient to provide an immune response. Rodents such as mice and rats represent commonly-employed animals. Spleen cells from the immunized animal are then fused with cells of an immortal myeloma cell. Where the immunized animal is a mouse, a representative myeloma cell comprises a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. This culturing provides a population of hybridomas from which specific hybridomas are selected. Typically, selection of hybridomas is performed by culturing the cells by single-clone dilution in microtiter plates, followed by testing the individual clonal supernatants for reactivity with an epitope. The selected clones can then be propagated indefinitely to provide the monoclonal antibody.

By way of specific example, to produce an antibody of the present invention, mice are injected intraperitoneally with between about 1-200 μg of an antigen comprising an epitope of the present invention. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. In one embodiment, the method of boosting and titering is repeated until a suitable titer is achieved. The spleen of the mouse with the highest titer is removed and the spleen lymphocytes are obtained by homogenizing the spleen with a syringe.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus “immortal”. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the present invention. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, and thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an epitope of the present invention. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the present invention in convenient quantity.

### LABORATORY EXAMPLES

The following Laboratory Examples have been included to illustrate representative and exemplary modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### Materials and Method for Laboratory Examples 1-4 Comparative ELISA

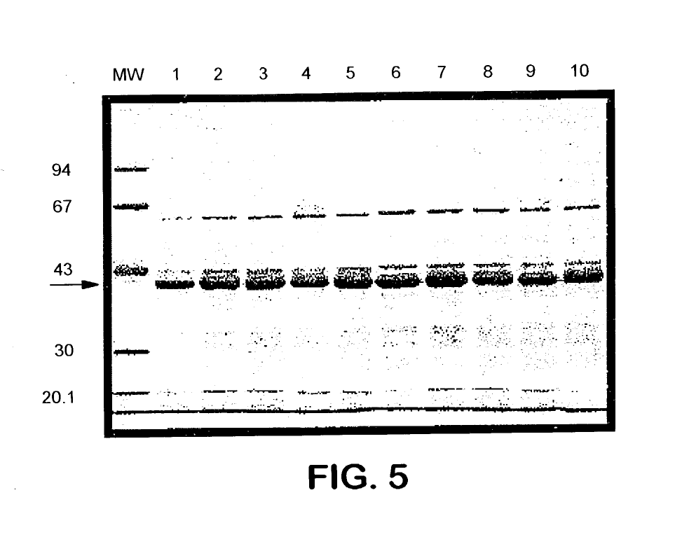

Anti-phospholipid scFv expression vectors were constructed, and scFv expression and purification were performed as previously described (Cocca et al., (1999) Prot. Expr. Purif. 17: 290-98). Purification of scFv by Ni-NTA affinity chromatography was performed as follows. Anti-phospholipid scFv were prepared by PCR amplification of the V_{H} and V_{L} coding regions, followed by cloning the pET26b+ derivative that contains the c-jun leucine zipper dimerization domain, the B domain of the Staphylococcus aureus protein A, and a penta-histidine tag (Cocca et al., (1999) Prot. Expr. Purif. 17: 290-98). The expression and purification of soluble scFv were performed as described hereinbelow. Briefly, ten milliliters of periplasmic extract, obtained by lysozyme digestion of the bacterial cell wall, were dialyzed overnight against binding buffer (e.g., 50 mM Tris-Cl, pH 8.0, 1.0 M NaCl, 10 mM imidazole), then mixed gently end-over-end with 1.0 ml packed Ni-NTA agarose (Qiagen, Inc. of Valencia, Calif.) on a Labquake™ rotator (Barnstead/Thermolyne, Inc. of Dubuque, Iowa) overnight at 4° C. The mixture was then applied to a poly-prep chromatography column (Bio Rad, Inc. of Hercules, Calif.), washed twice with 4.0 ml of wash buffer (e.g., 50 mM Tris-Cl, pH 8.0, 1.0 M NaCl, 40 mM imidazole, 0.5% Tween 20) and eluted with 2.0 ml of elution buffer (e.g., 50 mM Tris-Cl, pH 8.0, 1.0 M NaCl, 350 mM imidazole). The eluates containing purified scFv were dialyzed against phosphate buffered saline (PBS) and analyzed by SDS-PAGE and Coomassie blue staining. The SDS-PAGE gel is shown in FIG. 5. Lanes are marked as follows: MW, molecular weight marker; lane 1, R53G; lane 2, 157T; lane 3, D65G; lane 4, R53G/I57T/D65G; lane 5, 3H9; lane 6, S31R; lane 7, D56R; lane 8, N58R; lane 9, S76R; lane 10, D56R/S76R.

Immulon 2™ microtiter plates (Dynex Technologies, Inc. of Chantilly, Va.) were coated with DOPS (Sigma Chemical Co. of St. Louis, Mo.) at 10 μg/ml in ethanol and dried for 16 hours under vacuum. Plates were blocked with phosphate buffered saline containing 0.5% gelatin (PBS/gel) or PBS/gel with 10 μg/ml purified human β2GPI (Crystal Chem of Chicago, Ill.). Serial dilutions of scFv in PBS/gel were applied to wells, incubated for 1 hr, and unbound scFv were removed by washing. Bound scFv were detected with alkaline phosphatase-conjugated human serum IgG (Jackson Immunoresearch Laboratories of West Grove, Penn.). Following washing and addition of para-nitrophenyl phosphate (Sigma Chemical Co. of St. Louis, Mo.), absorbance was measured at 405 nm.

The inhibition assays were carried out using microtiter plates coated with DOPS and blocked with PBS/gel containing 10 μg/ml β2GPI. DOPS vesicles were prepared by drying under vacuum and hydrating the lipid in PBS, by vortexing. Vesicles were incubated with 200 μg/ml of β2GPI for 1 hour at room temperature. In parallel, 9 μg/ml of R53G/I57T/D65G or 2 μg/ml of D56R/S76R were incubated with increasing amounts of DOPS-β2GPI vesicles, calf thymus DNA (Sigma Chemical Co. of St. Louis, Mo.) digested to an average of 2 kb with S1 nuclease (Boehringer Mannheim of Indianapolis, Ind.), or PBS as a control. Following a 1 hour incubation, vesicles were removed from solution by centrifugation for 30 minutes at 14,000×g. The supernatant containing unbound scFv was applied to the wells containing DOPS-β2GPI and scFv were incubated for 1 hour. DNA-scFv complexes were not removed from solution prior to binding to DOPS-β2GPI. Unbound scFv were removed by washing and bound scFv were detected as described above.

### Flow Cytometry

Jurkat cells were harvested from culture, resuspended at a density of 10⁶ cells/ml in RPMI 1640 (Mediatech, Inc. of Herndon, Va.) containing 10% fetal bovine serum and 1.0 μM staurosporine (Sigma Chemical Co. of St. Louis, Mo.), and treated for 16 hours at 37° C. to induce apoptosis. Following treatment, 5×10⁵ cells were aliquoted into tubes and washed with 4.0 ml of ice-cold Hanks Balanced Salt Solution (Mediatech, Inc. of Herndon, Va.) containing 1.0 mM CaCl, 3% fetal bovine serum, and 0.02% NaN₃. Washed cells were incubated with 10 μg/ml of D56R/S76R, R53G/I57T/D65G, or 4V_{H}/1V_{L} (Seal et al., (2000) Arthritis Rheum. 43: 2132-2138) scFv for 15 minutes on ice and washed twice as above, followed by staining with FITC-conjugated Annexin V (BD Biosciences of San Diego, Calif.) and allophycocyanin-conjugated rabbit IgG (Molecular Probes of Eugene, Oreg.), as recommended by the manufacturers. Prior to analysis on a FACSCalibur (BD Biosciences of San Diego, Calif.), cells were stained with 5 μg/ml of propidium iodide (PI). Thirty thousand events were collected per sample and analyzed using FLOWJO™ software (Treestar, Inc. of San Carlos, Calif.).

### Statistical Analysis

Dose response curves were generated using GraphPad PRISM™ software (GraphPad Software, Inc. of San Diego, Calif.) Curve midpoints were determined by nonlinear regression with a variable slope and constant maximal absorbance value. Student's T test was employed to determine the statistical significance of the differences between binding curve midpoints.

### Molecular Modeling

The structure of the 3H9 scFv containing all forward mutations to arginine was modeled using the combined algorithm for antibody framework alignment and CDR loop homology modeling as described by Martin et al. (Martin, et al., (1989) Proc Natl. Acad. Sci. U.S.A. 86; 9268-9272). The optimized model coordinates were displayed using the Swiss-PDB Viewer.

### Laboratory Example 1

### Role of H Chain Somatic Mutations in Phosphatidylserine Recognition

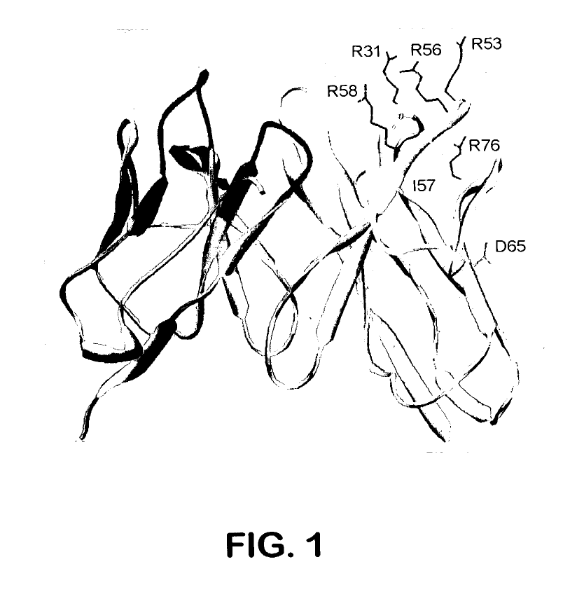

Following activation of a B cell, clonal expansion can be traced by features of antigen selection, including isotype switching and the accumulation of somatic mutations that increase the relative affinity for the antigen. By comparison to germline variable (V) genes, it was determined that the anti-DNA and anti-phospholipid autoantibody 3H9 is encoded by an immunodominant J553 V_{H} gene used repeatedly in autoantibodies from murine models of SLE (Radic & Weigert, (1994) Ann. Rev. Immunol. 12: 487-520). The 3H9 H chain acquired three somatic replacement mutations in CDR2: glycine to aspartic acid at position 65, threonine to isoleucine at position 57, and glycine to arginine at position 53 (FIG. 1). The role of these mutations in DNA binding has been tested previously (Radic et al., (1993) J. Immunol. 150: 4966-4977). To evaluate the role of mutations in shaping the antibody response to phosphatidylserine (e.g., DOPS), the mutations were reverted to germlne either individually or as a group, expressed as scFv fusion proteins in E. coli, and purified (FIG. 5).

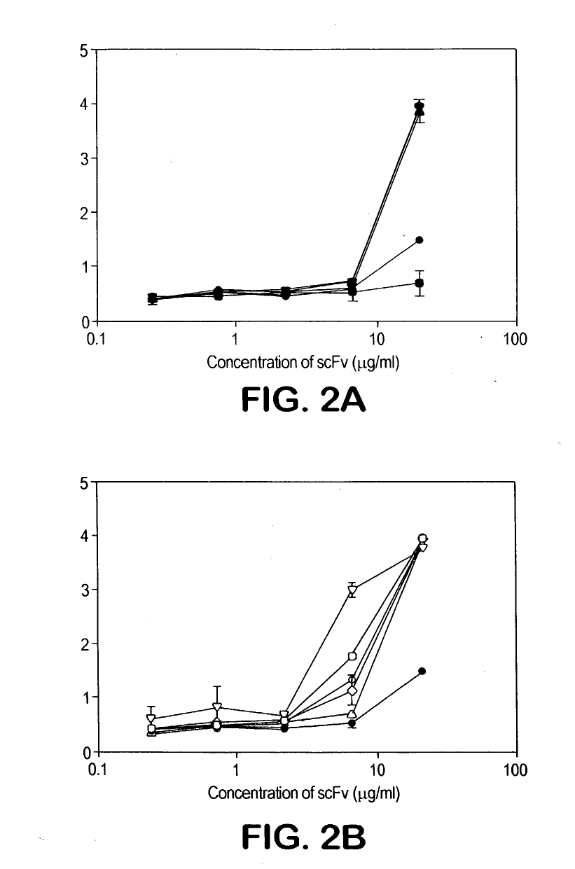

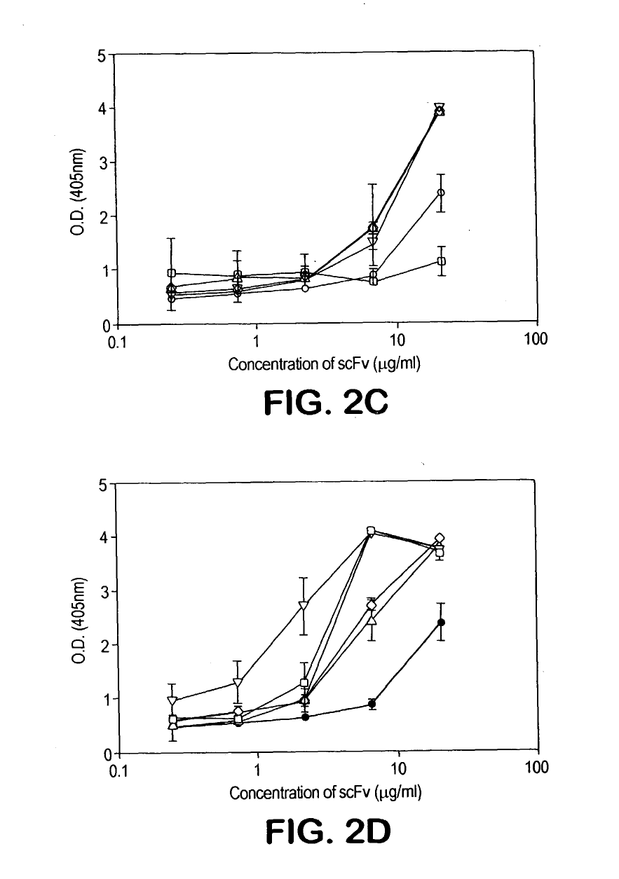

All scFv, except for the arginine to glycine revertant (R53G), exhibited detectable binding to DOPS, although 3H9 bound with low relative affinity (FIGS. 2A and 2B). Individual reversions of the isoleucine at position 57 (I57T) or the aspartic acid at position 65 (D65G) resulted in a three-fold increase in relative affinity compared to 3H9. The combined reversion of all three mutations also resulted in a three-fold increase in relative affinity. These results indicate that although the germline-encoded V_{H} is suitable for binding to phosphatidylserine, the somatic mutations that occurred during clonal expansion of 3H9 decreased this affinity. However, the mutation to arginine at position 53, a later event in the evolution of the B cell clone toward 3H9 (Shlomchik et al., (1987) Proc. Natl. Acad. Sci. U.S.A. 84: 9150-9154), resulted in an increased affinity for DOPS.

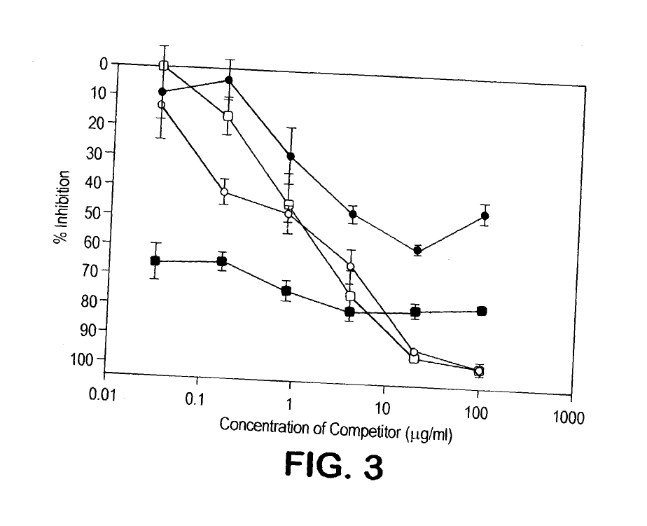

In addition to arginine 53, arginine residues at other sites within the CDR1, CDR2, and one unique location in the third framework region of anti-DNA H chain V genes create or enhance binding to DNA (Radic & Weigert, (1994) Ann. Rev. Immunol. 12, 487-520). Notably, arginine residues have also been observed at the same positions within the combining site of antibodies with dual reactivity with DNA and phospholipids (Kita et al., (1993) J. Immunol. 151: 849-856; Monestier et al., (1996) J. Immunol. 156: 2631-2641). To examine the role of arginine mutations in binding to DOPS, arginines were introduced at the equivalent sites within the H chain of 3H9 (FIG. 3). The single arginine mutations at positions 31 (S31R), 56 (D56R), 58 (N58R), and 76 (S76R) resulted in a three to four-fold increase in relative affinity compared to 3H9. In addition, the combination of D56R and S76R raised the relative affinity for DOPS six-fold above 3H9 and two-fold above each respective mutation alone. These results demonstrate that each of the arginines individually participates in binding to DOPS and that at least two of the arginines can be combined to produce an additive increase in DOPS binding.

### Laboratory Example 2

### Antibody Binding to DOPS is Enhanced by β2GPI

Antibodies to phospholipids often show enhanced binding to complexes of anionic phospholipids and β2GPI (McNeil et al., (1990) Proc. Natl. Acad. Sci. USA 87: 4120-4124). The role of β2GPI in the binding of scFv to DOPS, was investigated via binding assays that were performed in the presence of human β2GPI (FIGS. 2C and 2D).

The I57T, D65G, and germlne revertants, as well as 3H9, bound to DOPS-132GPI with significantly higher relative affinity than to DOPS alone, indicating that the binding of these variants is enhanced by β2GPI. The R53G mutant demonstrated no detectable binding to either DOPS-β2GPI or DOPS, confirming that the change to arginine at position 53 in 3H9 plays an important role in the recognition of DOPS-β2GPI as well as DOPS. Each of the forward mutants bound significantly better to DOPS-β2GPI than to DOPS. In addition, the combination of D56R and S76R mutations showed a greater increase in relative binding compared to 3H9 in the presence of β2GPI than in its absence. The results indicate that the combining sites of 3H9 and 7 of 8 variants conform more precisely to the complex between β2GPI and DOPS than to the phospholipid alone. Experiments using whole serum instead of purified β2GPI indicate that β2GPI is the main serum protein that enhances the binding of 3H9 and its variants to DOPS.

### Laboratory Example 3

### Effects of DNA on Antibody Recognition of Phosphatidylserine-β2GPI Complexes

The present data demonstrate that 3H9 and its variants share specificity for DOPS-β2GPI as well as DNA. To further define the dual binding specificity of these antibodies, competition experiments were carried out with DOPS-β2GPI vesicles and DNA (FIG. 3) using both the high affinity D56R/S76R and the germline revertant R53G/I57T/D65G scFv that binds poorly to dsDNA. The highest concentration of DNA (100 μg/ml) was able to completely inhibit the binding of both antibodies to DOPS-β2GPI, and lower concentrations showed approximately equivalent levels of inhibition for both antibodies.

In contrast, DOPS-β2GPI vesicles inhibited 45% of R53G/I57T/D65G and 80% of D56R/S76R binding to DOPS-β2GPI (FIG. 3). These results are consistent with DOPS-β2GPI ELISA results in that D56R/S76R has higher relative affinity for DOPS-β2GPI and is more sensitive to inhibition by lower concentrations of vesicles. The fact that DOPS-β2GPI vesicles did not completely inhibit binding to DOPS-β2GPI bound to the ELISA plate might indicate that the conformation of the DOPS-β2GPI complex in vesicles is not identical to the conformation on the ELISA plate and that the antibodies prefer the antigen as it is presented on the solid support.

### Laboratory Example 4

### Antibody Recognition of Apoptotic Cells

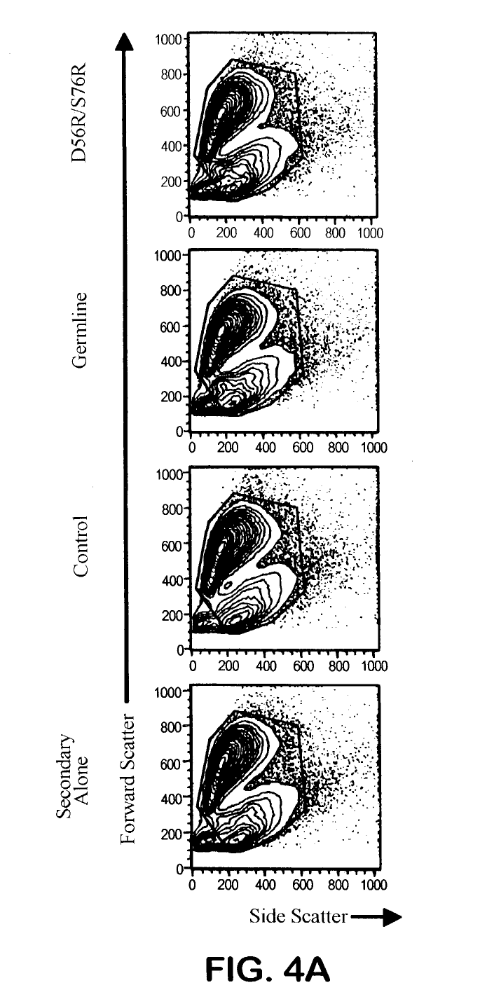

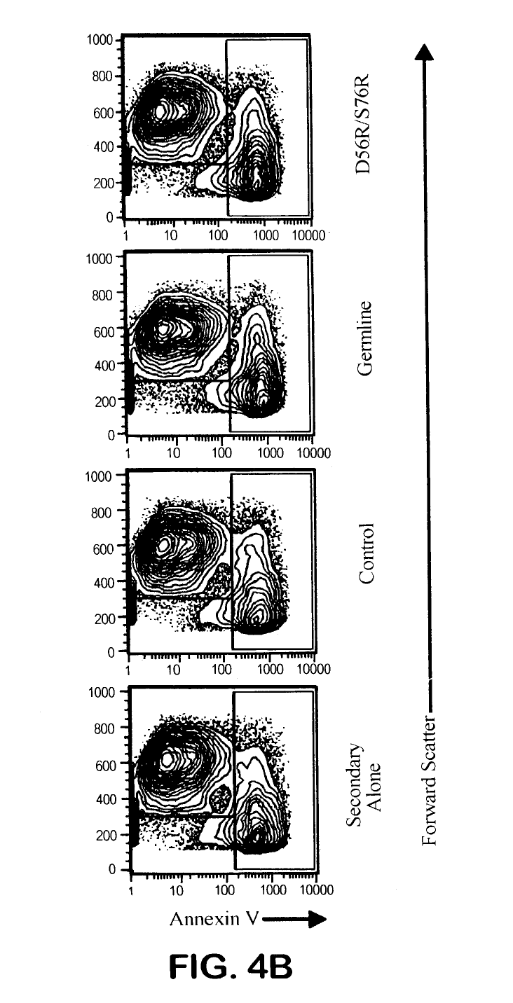

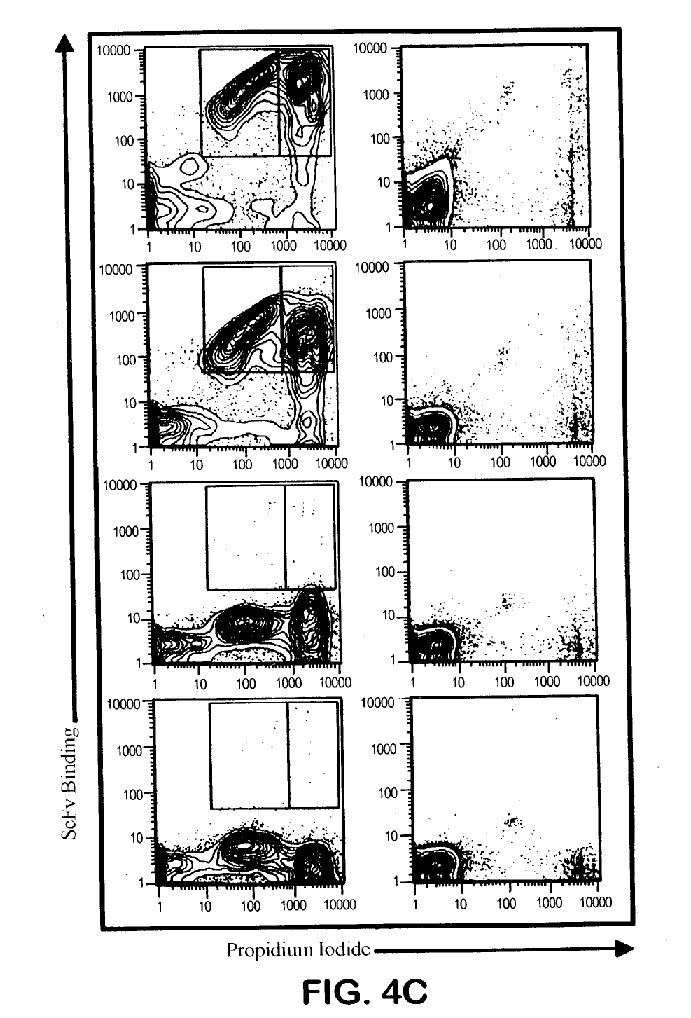

To evaluate the effects of somatic mutations on antibody recognition of apoptotic cells, binding of the two most diverse mutants, R53G1I57T/D65G and D56R/S76R, to Jurkat cells treated with staurosporine was assessed. Flow cytometry data were first gated according to forward and side scatter to exclude cell fragments and debris (FIG. 4A). Apoptotic cells were identified by staining with annexin V (FIG. 4B), and annexin V-positive and negative cells were analyzed separately to determine the extent of scFv binding and PI staining (FIG. 4C). Only annexin V-positive cells were bound by the R53G/I57T/D65G and D56R/S76R scFv, indicating that the 3H9 variants do not bind to the surface of viable cells. In contrast, the 4V_{H}/IV_{L} control scFv did not bind above background levels to either apoptotic or viable cells.

Among annexin V-positive cells, two populations of scFv-bound cells were distinguished based on PI staining intensity (FIG. 4C). These results indicate that the scFv used here preferentially recognize cells in the later phases of apoptosis. Consistent with the ELISA results, the mean fluorescence intensity (MFI) for D56R/S76R was approximately six-fold higher than for R53G/I57T/D65G and over one hundred-fold higher than for the control scFv (FIG. 4C). A two-fold decrease in MFI for the binding of D56R/S76R to cells cultured and washed in the absence of serum was observed, suggesting a role for β2GPI in the binding to apoptotic cells.

Since the scFv reacted only with cells that were bound by annexin V, a sensitive marker for phosphatidylserine, it is likely that variants of 3H9 bind to a cell surface epitope comprise or are similar in structure to, phosphatidylserine and β2GPI. Interestingly, approximately 5-10% of the annexin V-positive cells did not bind the R53G/I57T/D65G and D56R/S76R scFv. The lack of antibody binding to a subset of annexin V-positive cells might suggest differences in the accessibility or arrangement of phosphatidylserine. Such variations have been proposed to coincide with changes in lipid phase (Aguilar et al., (1999) J. Biol. Chem. 274: 25193-25196). Therefore, autoantibodies to phosphatidylserine-βGPI represent valuable probes for structural transitions that occur on the cell surface during apoptosis.

### Discussion of Laboratory Examples 1-4

To investigate the possible interaction of B cells with apoptotic cells, the structural requirements for autoantibody binding to phosphatidylserine, an anionic phospholipid expressed on the apoptotic cell surface, were analyzed (Savill & Fadok, (2000) Nature 407: 784-788; Fadok et al., (1992) J. Immunol. 148: 2207-2216). The disclosed results establish for the first time that the mouse germline encodes antibodies specific for phosphatidylserine (DOPS) (FIG. 2A), and that specificity for DOPS provides a possible explanation for the binding of these antibodies to apoptotic cells (FIG. 4C). Binding to DOPS is enhanced by β2GPI, a plasma protein that rapidly associates with anionic phospholipids on the membrane of apoptotic cells (Balasubramanian & Schroit, (1998) J. Biol. Chem. 273: 29272-29277; Price et al., (1996) J. Immunol. 157: 2201-2208). Complex formation between β2GPI and anionic phospholipids is associated with a structural transition in β2GPI (Wloch et al., (1997) J. Immunol. 159: 6083-6090) and correlates with increased immunogenicity (Krishnan et al., (1996) J. Immunol. 157: 2430-2439). The fact that 3H9 and its variants show higher relative affinity for DOPS-β2GPI than for DOPS alone indicates that some B cell receptors might recognize a protein-phospholipid complex that constitutes a unique structural feature of apoptotic cells.

The role of B cells whose surface receptors bind to apoptotic cells can serve a variety of functions. For example, Shaw et al. observed that T15 antibodies, long known for their protective role in responses to bacterial phosphorylcholine epitopes, also bind to apoptotic cells; Shaw et al. suggested that B cells with this specificity might serve “housekeeping” functions by removing cellular debris (Shaw et al., (2000) J. Clin. Invest. 105: 1731-1740). Thus, it is possible that immature B cells expressing V_{H}3H9 participate in the removal of apoptotic cell remnants. However, it is known that V_{H}3H9 plays a dominant role in the binding to DNA and phospholipids (Radic et al., (1991) J. Immunol. 146: 176-182; Ibrahim et al., (1995) J. Immunol. 155: 3223-3233; Seal et al., (2000) Eur. J. Immunol. 30: 3432-3440), that most V_{L} cannot block this binding (Ibrahim et al., (1995) J. Immunol. 155: 3223-3233), and that editing of V_{H} and/or V_{L} genes becomes obligatory. This is demonstrated by the fact that recombinase-deficient preB cells expressing the 3H9 V_{H} and V_{L} transgenes die by apoptosis (Xu et al., (1998) J. Exp. Med. 188: 1247-1254). Thus, if immature B cells participate in the uptake of apoptotic cell remnants in the bone marrow, at the same time, they must be undergoing receptor editing to ablate self-reactivity.

The two earliest replacement mutations, glycine at position 65 to aspartic acid and threonine at position 57 to isoleucine, greatly reduced the binding to DOPS-β2GPI, as seen from the comparison between the triple revertant and R53G. This observation might be related to the fact that central tolerance is largely intact in MRL/Ipr mice, as shown by studies with facultative self antigens (Rathmell & Goodnow, (1994) J. Immunol. 153: 2831-2842; Rubio et al., (1996) J. Immunol. 157: 65-71). In 3H9 transgenic mice bred on the MRL/Ipr genetic background, selection pressures result in an oligoclonal B cell expansion (Brard et al., (1999) J. Exp Med. 190: 691-704; Roark et al., (1995) J. Immunol. 154: 4444-4455), despite the abundant participation of the transgenes in the primary repertoire.

Somatic diversification mechanisms, such as V gene hypermutation and receptor editing, have the capacity to drastically alter the specificity of functionally rearranged Ig V genes. The L chain of 3H9, encoded by Vκ4/5Jκ4, could itself be the product of receptor editing by secondary VJ rearrangement, as Vκ4/5 genes are frequent editors in V_{H}3H9 H-chain-only transgenic mice (Radic et al., (1993) J. Exp. Med. 177: 1165-1173). It is possible that receptor diversification reduced the affinity of a 3H9 precursor for apoptotic cells, thus freeing it from central tolerance and allowing its exit from the bone marrow. This possibility is consistent with recent results from R53/I57T/D65G transgenic mice indicate that the 3H9 germline transgene also imposes stringent negative selection on B lymphocyte development and results in vigorous V_{L} receptor editing. Because R53G/I57T/D65G has a greater relative affinity for DOPS-β2GPI than for DNA, it is possible that binding to apoptotic cells provides a signal for negative selection of developing B cells that is perhaps as powerful as the binding to dsDNA.

In the periphery, the 3H9 clone might have encountered apoptotic cell remnants in the context of a dendritic cell. Studies by MacPherson and colleagues have shown transport of apoptotic cells to lymph nodes by dendritic cells (Huang et al., (2000) J. Exp. Med. 191: 435-444) and suggested a role for the association between newly emergent B cells and dendritic cells in the programming of isotype switching and antibody secretion (Wykes et al., (1998) J. Immunol. 161: 1313-1319). Such interactions might have selected for the replacement of glycine 53 with arginine and reinstated binding to DOPS-β2GPI. Alternatively, selection for binding to DNA or nucleoproteins might have provided a mechanism for recovering specificity for DOPS-β2GPI. In either case, the glycine 53 to arginine mutation greatly increased the relative affinity for ssDNA, dsDNA (Radic et al., (1993) J. Immunol. 150: 4966-4977) and DOPS-β2GPI (FIG. 2C), thus endowing 3H9 with dual specificity for DNA-protein complexes and apoptotic cells.

Dual specificity could have allowed the 3H9 clone to gain access to a variety of autoantigens, such as DNA, nucleosomes and ribonucleoproteins, that are sequestered in blebs and apoptotic bodies of dying cells (Casciola-Rosen, (1994) J. Exp. Med. 179: 1317-1330). Any B cell capable of binding and, possibly, internalizing such packets of autoantigens might have the potential to present a range of nuclear antigens to helper T cells. The initial interaction with a helper T cell can then determine the direction in which B cell specificity can evolve (Kaliyaperumal, (1996) J. Exp. Med. 183: 2459-2469), the nature of the retained replacement mutations, and the further course of affinity maturation.

Because arginine at position 53 of 3H9 plays a pivotal role in the dual specificity for DNA and DOPS-β2GPI, the role of additional arginine residues at positions 31 of CDR1, positions 56 and 58 of CDR2, and position 76 of FWR3 was examined. Each of those positions has been the site of somatic mutations to arginine in autoantibodies to DNA and phospholipids (Kita et al., (1993) J. Immunol. 151: 849-856; Monestier et al., (1996) J. Immunol. 156: 2631-2641). Previously, it has been reported that mutations to arginine at those positions raise the relative affinity of 3H9 for DNA (Radic et al., (1993) J. Immunol. 150: 4966-4977). Here, it was demonstrated that each mutation also increased the relative affinity for DOPS-β2GPI. In addition, inhibition experiments indicate that 56R and 76R serve equally well for DNA binding as for DOPS-β2GPI binding (FIGS. 2A-2D). Based on these results, it might be that B cells capable of binding to both the apoptotic cell surface and nuclear antigens have a higher probability of being selected than cells that are monospecific for only one of these antigens.

The characteristic of dual specificity is unusual because antibody specificity is generally focused toward the selecting antigen. Nevertheless, in autoimmunity, different combinations of autospecificities have been observed. Autoantibodies have been described that, in addition to DNA, bind to RNP or cytoskeletal elements (Radic et al., (1991) J. Immunol. 146: 176-182), to sub-nucleosome particles (Losman et al., (1992) Int. Immunol. 5: 513-523), to proteins involved in hnRNA splicing (Retter et al., (1996) J. Immunol. 156: 1296-1306), or to the hinge domain of the IgG molecule (Rumbley & Voss, (1995) Clin. Exp. Immunol. 102: 341-348). However, no antibodies have been reported that bind to DNA and to apoptotic cells. Ig receptors to apoptotic cell surface epitopes and DNA are unique in that they provide B cells with access to nuclear antigens, regardless of whether the nuclear antigens are sequestered in apoptotic blebs or not.

There is symmetry in the binding specificity of B cell receptors for DNA and apoptotic cells and pattern recognition receptors of the innate immune system. Macrophage scavenger receptors that participate in the clearance of apoptotic cells also bind to nucleic acids and anionic phospholipids (Pearson, (1996) Curr. Opin. Immunol. 8: 20-28). Although it is not the inventor's desire to be bound by any theory or mechanism, it is postulated that autoimmune B cells specific for apoptotic cell remnants could gain an advantage if they secrete antibodies that opsonize apoptotic cells and divert their uptake from scavenger receptors to Fc receptors. In that way, B cells that react with apoptotic cells might tip the balance from tolerance to autoimmunity.

### Materials and Methods for Laboratory Examples 5-8 Construction and Expression of an scFv

The construction of D56R/S76R was performed via the methods described herein above in Laboratory Examples 1-4. The 62.1 antibody is a member of clone 2 from the second hybridoma fusion involving mouse 384 (Krishnan et al., (1996) J. Immunol. 157: 2430-2439). The H chain CDR3 of 62.1 was obtained from mRNA by RT-PCR using the Access RT-PCR system, available from Promega of Madison, Wis. The cDNA incorporated the constant region primer Gamma2-1 (5′ CAGGGGCCAGTGCATAGA 3′) (SEQ ID NO: 5) and the flanking restriction endonuclease sites were introduced during the PCR step using the following primers: 38462FR3 (5′ TCTGAGGACTCCGGMKGTATTWCTGT 3′) (SEQ ID NO: 3) and JH4 Reverse (5′ ATCCCTGAGCTCACGGTGACTGAGGTTCC 3′) (SEQ ID NO: 4). The amplified CDR3 fragment was inserted between the Bsp El and Sac I restriction sites of the 3H9 heavy chain H3-filler cassette (Seal et al., (2000) Eur. J. Immunol. 30: 3432-3440) in the pET26b+ expression vector that also contained the 3H9 light chain, the c-jun leucine zipper, the protein A “B” domain, and a pentahistidine tag. The construct was confirmed by sequencing and named 3H9/62.1.

ScFv were purified via the methods described herein above in Laboratory Examples 1-4. Briefly, soluble scFv were recovered from the periplasm by digestion of the bacterial cell wall with lysozyme, dialyzed overnight against binding buffer (50 mM Tris-Cl, pH 8.0, 1.0 M NaCl, 10 mM imidazole), and then absorbed to 1.0 ml packed Ni-NTA agarose resin, available from Qiagen of Valencia, Calif., overnight at 4° C. The next morning, the slurry was applied to a chromatography column and washed twice with 4.0 ml of wash buffer (50 mM Tris-Cl, pH 8.0, 1.0M NaCl, 40 mM imidazole, 0.5% Tween 20). The purified scFv were eluted with 2.0 ml of elution buffer (50 mM Tris-Cl, pH 8.0, 1.0M NaCl, 350 mM imidazole), dialyzed overnight against PBS, and analyzed by SDS PAGE and Coomassie blue staining.

### Comparative ELISA

Binding of scFv to dioleoyl-phosphatidylserine (DOPS) as a complex with β2-glycoprotein I (β2GPI) and binding to biotinylated double stranded DNA was analyzed. In each assay, serial dilutions of scFv in triplicate were allowed to bind the antigen. For the DOPS-β2GPI assay, plates were washed with PBS and bound scFv were detected by incubation with alkaline phosphatase-conjugated human serum IgG, available from Jackson Immunoresearch Laboratories of West Grove, Pennsylvania. For the dsDNA assay, plates were washed and DNA that remained bound to the combining site of the scFv was detected by incubation with alkaline phosphatase-conjugated streptavidin (Jackson Immunoresearch Laboratories). For each assay, the absorbance resulting from the conversion of p-nitrophenol phosphate (PNPP) to p-nitorphenol (PNP) was measured at 405 nm.

### Cell Culture and Induction of Apoptosis

Jurkat cells were harvested from culture and resuspended at a density of 10⁶ /ml in RPMI 1640 containing 10% fetal bovine'serum (FBS). Apoptosis was induced for 12 hours with 1.0 μM staurosporine (Sigma Chemical Co. of St. Louis, Mo.), 2.0 μM camptothecin (Sigma), or 2.0 μg/ml anti-Fas antibody (clone 7C11; Beckman Coulter Inc. of Brea, Calif.). To inhibit apoptosis, parallel cultures were pre-incubated for 2 hours with 20 μM z-Val-Ala-Asp(Ome)-fluoromethylketone (z-VAD(Ome)-FMK) (Enzyme System Products of Livermore, Calif.). To inhibit membrane blebbing, parallel cultures were pre-incubated for 2 hours with 10 μM Y-27632 (Tocris of Ballwin, Mo.). At the end of the incubation period, 5×10⁵ cells were aliquoted into tubes and stained for flow cytometry or confocal microscopy.

### Flow Cytometry

Cells were washed with 4.0 ml of ice-cold Hanks Balanced Salt Solution (Mediatech, Herndon, Va.) containing 1.0 mM CaCl₂, 3% FBS, and 0.02% NaN₃ (HBSS/FBS). Washed cells were incubated with 10 μg/ml of D56R/S76R or 4Vh/1VI scFv for 15 minutes on ice, washed twice as above, and stained with FITC-conjugated Annexin V (BD Biosciences of San Diego, Calif.) and allophycocyanin-conjugated rabbit IgG (Molecular Probes of Eugene, Oreg.), as recommended by the manufacturers. 30,000 events were examined per sample on a FACSCalibur (BD Biosciences). The staining profiles were analyzed using FLOWJO™ software (Treestar, Inc. of San Carlos, Calif.).

### Confocal Microscopy

Cells were washed with HBSS and fixed with ice-cold 4% paraformaldehyde for 10 minutes. Fixed cells were washed with HBSS and incubated with 10 μg/ml of D56R/S76R, 3H9/62.1, or 4V_{H}/1V_{L} for 15 minutes on ice. After incubation with the scFv, cells were washed once with HBSS/FBS and incubated with biotinylated annexin V (BD Biosciences) for 15 minutes on ice. Cells were then washed twice with HBSS/FBS and stained with streptavidin-conjugated Alexaflour 488 (Molecular Probes), Rhodamine Red-conjugated human serum IgG (Jackson Immunoresearch Laboratories) and the nucleic acid dye TO-PRO-3 (Molecular Probes). Following staining for 15 minutes on ice, cells were washed with HBSS/FBS and mounted onto poly-L-lysine-coated glass slides for viewing with a Zeiss LSM 510 laser scanning microscope (Carl Zeiss, Inc. of Thorwood, N.Y.).

### Laboratory Example 5

### Binding of scFv to Cells in Apoptosis

The binding of the D56R/S76R scFv to Jurkat cells treated with staurosporine, camptothecin, or a murine anti-Fas monoclonal antibody (7C11) was examined. These treatments were chosen because they engage different pathways of apoptosis (Zhang et al., (1998) Nature 392: 296-300; Shao et al., (1999) EMBO J. 18: 1391-1406; Stepczynska et al., (2001) Oncogene 20: 1193-1202).

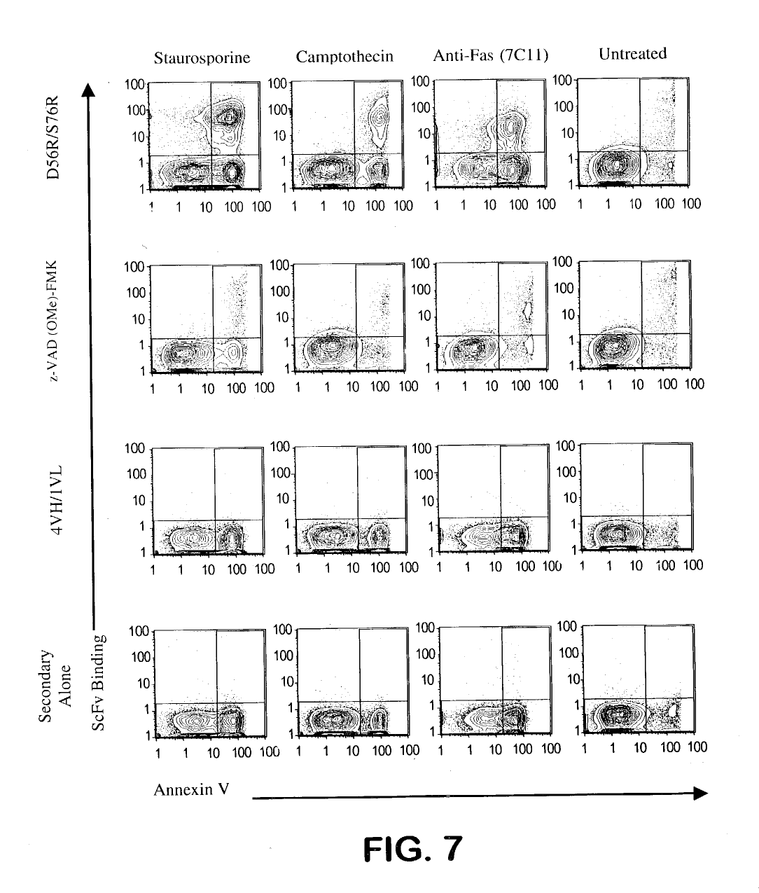

Flow cytometry was employed for the initial analyses because it provides a broad view of the entire cell population. Following 12 hr of incubation in the presence of apoptotic stimuli, apoptosis was examined by staining with annexin V, a molecule that recognizes phosphatidylserine in the presence of Ca⁺² (FIG. 7). Approximately 29% (camptothecin) to 62% (anti-Fas) of cells were observed to bind annexin V under these experimental conditions, whereas only 6% were positive for annexin V in the absence of any added stimuli. Annexin V positive cells could be further subdivided based on their binding to the D56R/S76R scFv. Between 10% (camptothecin) and 26% (staurosporine) of annexin positive cells expressed epitopes that were reactive with the scFv. The relative proportions of double positive cells were found to change with time of incubation: The proportion of annexin V and D56R/S76R scFv double-positive cells from among the total of annexin V positive cells gradually increased from less than 3% at 4 hr (not shown) to the values observed at 12 hr (FIG. 7).

The binding of the scFv was dependent on the effective induction of apoptosis, as pretreatment of the cells with z-VAD(OMe)-FMK, a broad inhibitor of caspases, largely eliminated binding of the scFv (FIG. 7). As expected, in the presence of this inhibitor, the staining with annexin V also decreased to near-background levels. The binding of the scFv to apoptotic cells was mediated by the combining site of D56R/S76R because the presence of a human anti-DNA derived scFv, 4V_{H}/1V_{L} (Seal et al., (2000) Arthritis Rheum. 43: 2132-2138.), did not result in apparent binding to either annexin V-positive or annexin V-negative Jurkat cells.

### Laboratory Example 6

### Microscopic Localization of Binding

To gain a more detailed view of the site of scFv binding, immunofluorescence was employed to examine the interaction between annexin V, scFv, and Jurkat cells treated to induce apoptosis.

Confocal immunofluorescence microscopy with D56R/S76R or 4VH/1VL scFv, annexin V, and TO-PRO-3, a DNA intercalating dye was also performed. Each of the three treatments for inducing apoptosis, camptothecin, staurosporine, or anti-Fas, resulted in similar staining morphologies.

Cells in early to mid apoptosis stained only faintly with annexin V and were larger than the remaining cells that stained brightly with annexin V. ScFv bound avidly to cells that stained brightly with annexin V. The coincident binding of the two molecules could be visualized as patches of yellow, due to the overlap between the Alexafluor 488 signal associated with annexin V and the Rhodamine Red fluorescence used to localize the scFv. The annexin V positive cells represent the two populations of annexin V positive cells identified by flow cytometry (FIG. 7): one population that predominantly stained with annexin V, and a second population that stained with both annexin V and scFv.

Cells in more advanced apoptosis were bound by annexin V and the D56R/S76R scFv. The two molecules did not occupy identical positions on the cell surface but instead were increasingly localized to non-overlapping membrane domains. This was particularly evident at later stages of apoptosis.

The segregation of the ligands for annexin V from the epitopes recognized by D56R/S76R was most evident in cells undergoing blebbing. For example, cells that exhibited several large surface blebs that were bound by the D56R/S76R scFv. On these cells, areas of overlap between annexin V and the scFv were limited. In contrast to D56R/S76R, annexin V occupied more central areas of the cell that extended between adjacent blebs. In addition, a few smaller blebs primarily stained with annexin V. On cells in earlier stages of apoptosis, in which annexin V staining was already well developed, scFv binding was barely detectable. In cases of limited binding by the scFv, binding was concentrated to focal areas that appeared to coincide with annexin V staining.

Of the five cells that stained with annexin V only two were also bound by the scFv. In each case, scFv binding was only observed over areas of the cell interior that also stained with the DNA intercalator TO-PRO-3. This suggests that the 4V_{H}/1V_{L} scFv did not bind membranes of apoptotic cells. Instead, 4V_{H}/1V_{L} bound chromatin or DNA in fragmented nuclei that characterize a relatively late stage of apoptosis.

In the absence of scFv binding no Rhodamine Red staining of apoptotic cells was observed. It was also observed that pretreatment with z-VAD(OMe)-FMK reduces both annexin V and scFv binding to background levels despite treatment with staurosporine, and it was observed that scFv and annexin V did not bind to live cells. Although not shown, z-VAD(OMe)-FMK also greatly reduced staining of cells treated with camptothecin or anti-Fas antibody, thus indicating that D56R/S76R binding requires progress through the execution phase of apoptosis.

### Laboratory Example 7

### Comparison Between Two scFv that Bind Cells in Apoptosis

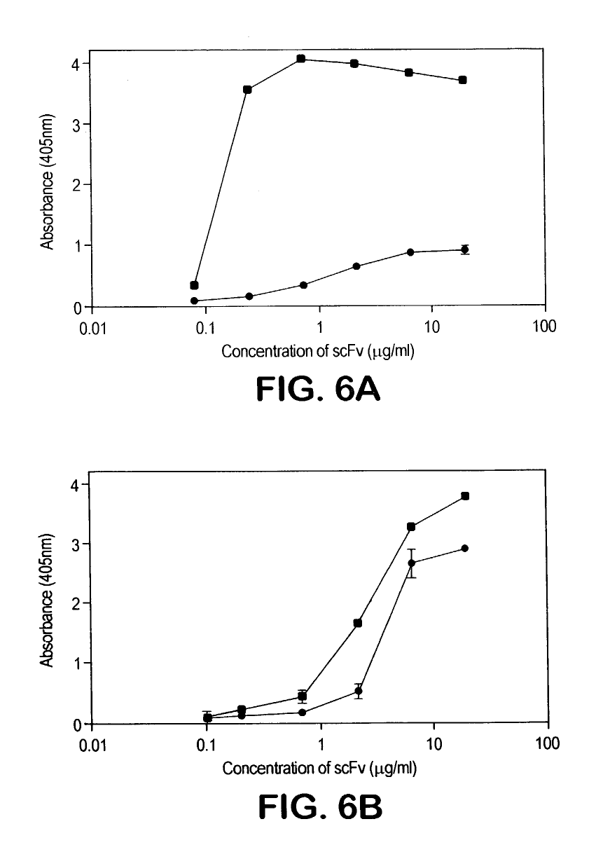

To investigate the possibility that other derivatives of 3H9 might react with cells in apoptosis, experiments took into account the observation that an exchange of the CDR3 domain of 3H9 for a different CDR3 eliminates DNA binding, yet maintains specificity for cardiolipin (Seal et al., (2000) Eur. J. Immunol. 30: 3432-3440), an anionic phospholipid that is mobilized from the mitochondrial membrane during apoptosis (Sorice et al., (2000) Clin Exp. Immunol. 122: 277-284). The 3H9/62.1 scFv was constructed by exchanging the CDR3 of the anti-DNA 384s clone 2 #62 (Krishnan et al., (1996) J. Immunol. 157: 2430-2439) for the CDR3 found in 3H9 (Seal et al., (2000) Eur. J. Immunol. 30: 3432-3440). The 3H9/62.1 and D56R/S76R scFv bound with nearly identical relative affinities to dioleoyl-phosphatidylserine-β2GPI (FIG. 3), an in vitro analogue of a complex antigen found on the surface of apoptotic cells (Price et al., (1996) J. Immunol. 157: 2201-2208.). In contrast, the binding of the 3H9/62.1 scFv to dsDNA was drastically reduced (FIG. 6B).

In confocal fluorescence microscopy, 3H9/62.1 preferentially bound annexin V-positive cells, although not all annexin V-positive cells were bound by the scFv. The initial site of 3H9/62.1 binding coincided with annexin V-positive domains of the cell surface and later tended to localize to protrusions from the cell membrane. Increased staining tended to focus on a number of smaller protrusions of the cell surface. In sum, both 3H9/62.1 and D56R/S76R (FIG. 8) bound preferentially to the surface of well-developed apoptotic blebs, and blebbing coincided with increasing segregation between the domains recognized by annexin V and either of the two scFv.

### Laboratory Example 8

### Nuclear Fragmentation, Blebs and Apoptotic Bodies

Both the formation of apoptotic blebs and the fragmentation of the nucleus are dependent on the activation of the Rho-associated kinase ROCK I (Coleman et al., (2001) Nature Cell Biol. 3: 336-345; Sebbagh et al., (2001) Nature Cell Biol. 3: 346-352). For this kinase, a highly specific inhibitor, Y-27632, is available (Narumiya et al., (2000) Method Enzymol. 325: 273-284). Cells were treated with anti-Fas in the presence or absence of this inhibitor and examined by microscopy. scFv was observed to bind to cells that were in the later stages of apoptosis, as indicated by the permeability of their plasma membrane to TO-PRO-3. It is also possible that the cells became permeable as a result of mechanical damage induced by manipulations inherent in the technique used. In such cases, binding of the D56R/S76R scFv mostly coincided with blebs that contained fragments of nuclear material. A close-up view of one of those blebs shows, in an optical section, the dense packing of the bleb interior by TO-PRO-3-reactive material and the contiguous staining of the bleb surface by the scFv. In addition, we noted the binding of D56R/S76R to blebs that did not contain nuclear material. These less frequently observed surface structures could be analogous to blebs that contain cytoplasmic ribonucleoprotein complexes (Casciola-Rosen et al., (1994) J. Exp. Med. 179: 1317-1330).

In cells treated with Y-27632, blebbing and nuclear fragmentation was inhibited and the binding of the D56R/S76R scFv was greatly reduced. This indicates that binding of the scFv is not a default result of apoptosis progression, but that it requires enzyme activity and occurs in concert with the fragmentation of the nucleus and the migration of nuclear domains to sites of plasma membrane blebbing. On occasion, blebs appeared to separate from the remainder of the cell, perhaps indicating a link between blebbing, the formation of apoptotic bodies, and hypodiploid DNA content that characterize apoptosis.

### Discussion of Laboratory Examples 5-8

The observation that characteristic SLE autoantigens are packaged into blebs at the surface of apoptotic cells (Casciola-Rosen et al., (1994) J. Exp. Med. 179: 1317-1330.) has fostered interest in the study of apoptotic cells as the source of autoantigens. However, it is not clear what conditions facilitate B cell reactivity with apoptotic cells. It has been demonstrated above in accordance with the present invention that autoantibodies with dual specificity for phosphatidylserine and DNA bind to apoptotic cells. In a continued effort to understand the interaction between B cells and apoptotic cells, Example 5-8 demonstrate that recombinant antibodies recognize a unique cell surface epitope that is expressed on Jurkat cells exposed to three distinct death signals.

An early event in the chronology of apoptosis that can be detected by the binding of annexin V is the exposure of phosphatidylserine on the outer membrane leaflet (Martin et al., (1995) J. Exp. Med. 182: 1545-1556; Vermes et al., (1995) J. Immunol. Methods. 184: 39-51). Despite the fact that D56R/S76R also can recognize phosphatidylserine in vitro, it was found that the epitope for the antibody is not identical to the ligand for annexin V (FIG. 7), and that each molecule bound unique as well as overlapping locations on the cell. These results suggest that annexin V and D56R/S76R recognize distinct molecules on the apoptotic cell surface or that they differentiate among distinct conformations of phosphatidylserine.

The expression of the epitope recognized by D56R/S76R depends on the activation of caspases, as treatment with z-VAD(OMe)-FMK eliminated binding of the antibody as well as annexin V to the cells (FIG. 7). The sequential activation of caspases results in the cleavage of various death substrates, many of which are targets of autoantibodies in SLE. The sequential activation of caspases also results in the generation of characteristic morphologic changes in the cell, including cytoplasmic and nuclear condensation, fragmentation of the cell nucleus, and blebbing of the cell membrane (Hengartner, (2000) Nature 407: 770-776; Häcker, (2000) Cell Tissue Res. 301: 5-17). Since the formation of blebs and the packaging of fragmented nuclear material into them depends on the activity of the enzyme ROCK I (Coleman et al., (2001) Nature Cell Biol. 3: 336-345; Sebbagh et al., (2001) Nature Cell Biol. 3: 346-352.), the effects of a specific inhibitor of ROCK I on the binding of D56R/S76R to apoptotic cells were evaluated. As previously observed (Coleman et al., (2001) Nature Cell Biol. 3: 336-345), the inhibitor Y-27632 did not affect binding of annexin V. In contrast, Y-27632 decreased antibody binding, concomitant with decreased blebbing, indicating that blebs play a central role in the binding of D56R/S76R to apoptotic cells.

Given that D56R/S76R binds tightly to DNA and nucleosomes (Radic et al., (1993) J. Immunol. 150: 4966-4977) as well as to phosphatidylserine (FIGS. 6A and 6B), and that cells might express receptors for DNA or chromatin on their surface (Siess et al., (2000) J. Biol. Chem. 275: 33655-33662), the possibility existed that the binding to apoptotic cells by D56R/S76R might be mediated by nucleic acids or chromatin released from cells in culture. To exclude this possibility, the binding of D56R/S76R to apoptotic cells treated with DNAse I was evaluated. It was observed that binding of this antibody was not affected by the enzyme treatment. A more definitive observation against the participation of DNA was provided by generating the CDR3 variant of 3H9, 3H9/62.1, which retains specificity for phosphatidylserine, but has much lower relative affinity for DNA (FIGS. 6A and 6B). Comparison of the binding of D56R/S76R and 3H9/62.1 to apoptotic cells revealed that the different scFv bind similarly to the cell surface. Together, the results of these experiments indicate that binding to apoptotic cells is not mediated by DNA. However, our results do not preclude the possibility that the antibodies can recognize other antigens, including the putative C1q receptor or the C-reactive protein, proteins that play a role in scavenger cell clearance of apoptotic cells (Taylor et al., (2000) J. Exp. Med. 192:359-366; Gershov et al., (2000) J. Exp. Med. 192:1353-1363).

In general, apoptotic cells are quickly cleared by scavenger cells via a variety of receptors on the phagocyte (Fadok et al., (2000) Nature. 405: 85-90; Platt et al., (1996) Proc. Natl. Acad. Sci. U.S.A. 93: 12456-12460; Fukasawa et al., (1996) Exp. Cell. Res. 222: 246-250; Savill et al., (1990) Nature 343: 170-174; Devitt et al., (1998) Nature 392: 505-509; Ren et al., (1995) J. Exp. Med. 181: 1857-1862). This rapid removal suggests that phagocytosis occurs prior to cells initiating blebbing. However, because the binding of c1q is specific for blebs and apoptotic bodies (Navratil et al., (2001) J. Immunol. 166: 3231-3239), at least occasionally cells can proceed to the blebbing stage of apoptosis in vivo. This is implied by the observation that C1q-deficient mice develop characteristic anti-nuclear antibodies (Botto et al., (1998) Nature Genet. 19: 56-59) and exhibit delayed clearance of apoptotic cells (Taylor et al., (2000) J. Exp. Med. 192: 359-366).

It is possible that an antibody that competes with components of complement for the binding to blebs on apoptotic cells can bring about a situation analogous to complement deficiencies, whereby the normal removal of apoptotic remnants is disrupted, and the likelihood increases that additional autoreactive B cells can encounter apoptosis-related autoantigens. This situation may favor uptake by and positive selection of autoreactive B cells as an overture for autoimmune disease.

### Laboratory Example 9

### Visualization of scFv with Epitope Associated with Surface Blebs

Cells were washed with HBSS and fixed with ice-cold 4% paraformaldehyde for 10 minutes. Fixed cells were washed with HBSS/FBS and incubated with 10 μg/ml of D56R/S76R, for 15 minutes on ice. After incubation with the scFv, cells were washed once with HBSS/FBS and incubated with biotinylated annexin V (BD Biosciences) for 15 minutes on ice. Cells were washed twice with HBSS/FBS and stained with streptavidin-conjugated Alexaflour 488 (Molecular Probes), Rhodamine Red-conjugated human serum IgG (Jackson Immunoresearch Laboratories) and the nucleic acid dye TO-PRO-3 (Molecular Probes). After a 15 minute incubation on ice, cells were washed with HBSS/FBS and mounted onto poly-L-lysine-coated glass slides for viewing with a Zeiss LSM 510 laser scanning microscope (Carl Zeiss Inc., Thorwood, N.Y.). A micrograph of the results of this procedure is depicted in FIG. 8.

FIG. 8 shows that D56R/S76R binds to blebs that contain fragments of the nucleus (large arrowheads) as well as to blebs that do not contain nuclear material (small arrowheads). Binding of scFv and annexin V is largely segregated, in that annexin V binds between blebs. Most blebs bound by the scFv contain pieces of the fragmented nucleus and are stained by TO-PRO3, a DNA binding dye, although it is not necessary for cells to be permeable to TO-PRO-3, in order to react with the scFv.

### REFERENCES

The references listed below as well as all references cited in the specification are incorporated herein by reference to the extent that they supplement, explain, provide a background for or teach methodology, techniques and/or compositions employed herein.

Abrams et al., (1993) Development 117: 29-44

Aguilar et al., (1999) J. Biol. Chem. 274: 25193-25196

Altschul et al., (1990) J. Mol. Biol. 215: 403-10

Arends & Wyllie, (1991) Int. Rev. Exp. Pathol. 32: 223-549

Balasubramanian & Schroit, (1998) J. Biol. Chem. 273: 29272-29277

Bertrand et al., (1993) Exp. Cell Res. 207: 388-397

Bhalla et al., (1992) Blood 80: 2883-2390

Bhalla et al., (1993) Blood 82: 3133-3140

Botto et al., (1998) Nature Genet. 19: 56-59

Brard et al., (1999) J. Exp Med. 190: 691-704

Campana et al., (1993) Leukemia 7: 482

Casciola-Rosen et al., (1994) J. Exp. Med. 179: 1317-1330

Casciola-Rosen, (1994) J. Exp. Med. 179: 1317-1330

Casey et al., (1995) J. Immunol. Meth. 179: 105-116

Chiron et al., (1992) Blood 80: 1307

Cocca et al., (1999) Protein Expres. Purif. 17: 290-298

Cohen et al., (1992) Biochem. J. 286: 331-334

Coleman et al., (2001) Nature Cell Biol. 3: 336-345

Crompton et al., (1992) Biochem. Biophys. Res. Commun. 183: 532-537

Devitt et al., (1998) Nature 392: 505-509

Ellis et al., (1991) Annu. Rev. Cell. Biol. 7: 663-698

Fadok et al., (1992) J. Immunol. 148: 2207-2216

Fadok et al., (2000) Nature. 405: 85-90

Falcieri et al., (1993) Biochem. Biophys. Res. Commun. 193: 19 (1993)

Frey, (1995) Cytometry 21: 265-274

Fukasawa et al., (1996) Exp. Cell. Res. 222: 246-250

Gavrieli et al., (1992) J. Cell Biol. 119: 493-501

Gershov et al., (2000) J. Exp. Med. 192:1353-1363

Gougen & Montagnier, (1993) Science 260: 1269-1270

Grether et al., (1995) Gene Dev. 9: 1694-1708

Gribskov & Burgess, (1986) Nucl. Acids. Res. 14: 6745-6763

Gunji et al., (1991) Cancer Res. 51: 741-743

Häcker, (2000) Cell Tissue Res. 301: 5-17

Hafifer et al., (1995) Curr. Opin. Genet. Dev. 5:84-90

Hay et al., (1994) Development 120: 2121-2129

Hefeneider et al., (1990) J. Invest. Dermatol. 94: 79S-84S

Hengartner, (2000) Nature 407: 770-776

Henikoff& Henikoff, (1989) Proc. Natl. Acad. Sci. U.S.A. 89:10915

Hockenbery et al., (1990) Nature 348: 334-336

Huang et al., (2000) J. Exp. Med. 191: 435-444

Hurle, (1988) Meth. Achiev. Exp. Pathol. 13: 55-86

Ibrahim et al., (1995) J. Immunol. 155: 3223-3233

Jacobson et al., (1993) Nature 361: 365-369

Janson & Rydén (eds), (1998) Protein Purification: Principles, High Resolution Methods, and Applications (2^{nd} ed.), Wiley-Liss, New York, N.Y.

Kaliyaperumal, (1996) J. Exp. Med. 183: 2459-2469

Karlin & Altschul, (1993) Proc. Natl. Acad. Sci. U.S.A. 90: 5873-5887

Karp et al., (1994) Blood 83: 517-530

Kerr & Harmon, (1991) in Apoptosis: The Molecular Basis of Cell Death, pp. 5-29, Cold Spring Harbor Laboratory Press, New York

Kerr et al., (1972) Br. J Cancer. 26: 239-257

Kita et al., (1993) J. Immunol. 151: 849-856

Krishnan et al., (1996) J. Immunol. 157: 2430-2439

Kroemer et al., (1995) FASEB J. 9: 1277-1287

Kyte & Doolittle, (1982) J. Mol. Biol. 157: 105-132

Lassmann et al., (1995) Acta Neuropathol. 89: 35-41

Li et al., (1994) Leukemia Lymphoma 13: 65

Liston et al., (1996) Nature 379: 349-353

Losman et al., (1992) Int. Immunol. 5: 513-523

Lotem & Sachs, (1992) Blood 80: 1750-1757

Martin et al., (1989) Proc. Natl. Acad. Sci. U.S.A. 86: 9268-9272

Martin et al., (1995) Cell 82: 349-352

Martin et al., (1995) J. Exp. Med. 182: 1545-1556

McNeil et al., (1990) Proc. Natl. Acad. Sci. USA. 87: 4120-4124

Meyaard et al., (1992) Science 257: 217-219

Miyashita & Reed, (1993) Blood 81:151-157

Monestier et al., (1996) J. Immunol. 156: 2631-2641

Narumiya et al., (2000) Method Enzymol. 325: 273-284

Navratil et al., (2001) J. Immunol. 166: 3231-3239

Needleman & Wunch, (1970) J. Mol. Biol. 48: 443-453

Nicoletti et al., (1991) J. Immunol. Methods 139: 271-279

Nitatori et al., (1995) J. Neurosci. 15: 1001-1011

O'Shea et al., (1989) Science. 245: 646-648

Oltvai et al., (1994) Cell 79: 189-192

Oppenheim, (1991) Ann. Rev. Neurosci. 14: 453-501

Pack & Plückthun, (1992) Biochem. 31: 1579-1584

Pearson, (1996) Curr. Opin. Immunol. 8: 20-28

Platt et al., (1996) Proc. Natl. Acad. Sci. U.S.A. 93: 12456-12460

Plückthun, (1994) The Pharmacology of Monoclonal Antibodies, vol. 113, (Rosenburg and Moore eds.), Springer-Verlag, New York, N.Y., pp.269-315

Price et al., (1996) J. Immunol. 157: 2201-2208

Pronk et al., (1996) Science 271: 808-810

Rabizadeh et al., (1993) J. Neurochem. 61: 2318-2321

Radic & Seal, (1997) Methods 11: 20-26

Radic & Weigert, (1994) Ann. Rev. Immunol. 12, 487-520

Radic et al., (1991) J. Immunol. 146:176-182

Radic et al., (1993) J. Exp. Med. 177: 1165-1173

Radic et al., (1993) J. Immunol. 150: 4966-4977

Raff, (1992) Nature 356: 397-400

Rathmell & Goodnow, (1994) J. Immunol. 153: 2831-2842

Ren et al., (1995) J. Exp. Med. 181: 1857-1862

Retter et al., (1996) J. Immunol. 156: 1296-1306

Roark et al., (1995) J. Immunol. 154: 4444-4455

Roy et al., (1995) Cell 80: 167-178

Rubio et al., (1996) J. Immunol. 157: 65-71

Rumbley & Voss, (1995) Clin. Exp. Immunol. 102: 341-348

Sambrook et al., (1989) Molecular Cloning: A Laboratory Manual, Cold Spring Harbor Laboratory Press, Cold Spring Harbor, Cold Spring Harbor, N.Y.

Saunders & Fallon, (1967) Cell Death in Morphogenesis, Major Problems in Developmental Biology; pp. 289-314, Academic Press, New York, N.Y.

Savill & Fadok, (2000) Nature 407: 784-788

Savill et al., (1990) Nature 343: 170-174 Schulze-Osthoff et al., (1994) J. Cell Biol. 127:15-20

Schwartz & Dayhoff, (1978) in Atlas of Protein Seguence and Structure (Dayhoff, ed.) Washington, D.C.: National Biomedical Research Foundation, 5: 353-358

Seal et al., (2000) Arthritis Rheum. 43: 2132-2138

Seal et al., (2000) Eur. J. Immunol. 30: 3432-3440

Sebbagh et al., (2001) Nature Cell Biol. 3: 346-352

Shao et al., (1999) EMBO J. 18: 1391-1406

Shaw et al., (2000) J. Clin. Invest. 105: 1731-1740

Shlomchik et al., (1987) Proc. Natl. Acad. Sci. U.S.A. 84: 9150-9154

Smith & Waterman, (1981) Adv. In Appl. Math. 2: 482-489

Smith et al., (1994) Cell 76, 959-962

Sorice et al., (2000) Clin Exp. Immunol. 122: 277-284

Steller, (1995) Science 267: 1445-1449

Stepczynska et al., (2001) Oncogene20: 1193-1202

Sugimoto et al., (1994) EMBO J 13: 2023-2028

Tan et al., (1966) J. Clin. Invest. 45: 1732-1740

Tartaglia et al., (1992) Immunol. Today 13: 151-153

Taylor et al., (2000) J. Exp. Med. 192: 359-366 Thompson, (1995) Science 267: 1456-1462

Truman, (1984) Ann. Rev. Neurosci. 7: 171-188

Vaux et al., (1994) Cell 76: 777-779 Vermes et al., (1995) J. Immunol. Methods 184: 39-51

Vito et al., (1996) Science 271: 521-525)

Wellner, (1971) Anal. Chem. 43: 597

Wetmur & Davidson, (1968) J. Mol. Biol. 31: 349-70

White et al., (1996) Science 271: 805-807

Wijsman et al., (1993) J. Histochem. Cytochem. 41: 7-12

Williams, (1991) Cell 65: 1097-1098

Woo, (1995) N. Engl. J. Med. 333:18-25

Wood et al., (1993) Neuron 11: 621-632

Wykes et al., (1998) J. Immunol. 161: 1313-1319

Wyllie et al., (1980) Int. Rev. Cytol. 68: 251-306

Wyllie et al., (1984) J. Pathol. 142: 67-77

Wyllie et al., (1998) Int. Rev. Cytol. 68: 251-306

Wyllie, (1980) Nature 284:555-556

Xu et al., (1998) J. Exp. Med. 188: 1247-1254

Zhang et al., (1998) Nature 392: 296-300

Zwelling et al., (1993) Biochem. Pharmacol. 45: 516

U.S. Pat. No. 4,016,043

U.S. Pat. No 4,196,265

U.S. Pat. No.4,554,101

U.S. Pat. No. 5,260,203

U.S. Pat. No. 5,935,801

U.S. Pat. No. 6,048,703

PCT publication WO86/01533

British Patent No. GB2188638

It will be understood that various details of the invention may be changed without departing from the scope of the invention. Furthermore, the foregoing description is for the purpose of illustration only, and not for the purpose of limitation.

What is claimed is:

1. An isolated antibody composition, comprising a 3H9 antibody-derived variable region that specifically recognizes an epitope on the surface of an apoptotic cell, the epitope being detectable in cells undergoing apoptosis and undetectable in cells not undergoing apoptosis.

2. The antibody composition of claim 1, wherein the variable region is further defined as a single chain variable fragment (scFv).

3. The antibody composition of claim 2, wherein the scFv comprises the amino acid sequence of one of SEQ ID NOs: 6 and 7.

4. The antibody composition of claim 2, wherein the scFv comprises one or more of a variable segment of an antibody heavy chain, a variable segment of an antibody light chain, a linker sequence, a dimerization domain, a purification sequence and combinations thereof.

5. The antibody composition of claim 4, wherein the heavy chain comprises a heavy chain of a 3H9 antibody.

6. The antibody composition of claim 4, wherein the light chain comprises a light chain of a 3H9 antibody.

7. The antibody composition of claim 4, wherein the dimerization domain is a leucine zipper.

8. The antibody composition of claim 4, wherein the leucine zipper is one or more of a c-fos leucine zipper, a c-jun leucine zipper and combinations thereof.

9. The antibody composition of claim 4, wherein the purification sequence is selected from the group consisting of the B domain of a protein A, a histidine tag and combinations thereof.

10. The antibody composition of claim 1, wherein the epitope is present in a complex comprising phosphatidylserine, dioleoyl phosphatidylserine, β2GPI, a nucleoprotein, a constituent of an apoptotic cell surface and combinations thereof.

11. The antibody composition of claim 1, wherein the epitope is located in a region of a bleb formed on the surface of an apoptotic cell.

12. The antibody composition of claim 1, further comprising a detectable moiety.

13. The antibody composition of claim 12, wherein the detectable moiety is selected from the group consisting of a radiolabel, a fluorescent label, a chemiluminescent label and an enzyme.

14. A dimer comprising an antibody composition of claim 2.

15. An isolated and purified polynucleotide encoding an antibody polypeptide, the polynucleotide comprising one or more of a polynucleotide encoding a variable segment of a heavy chain of an antibody, a polynucleotide encoding a variable segment of a light chain of an antibody, a polynucleotide encoding a linker sequence, a polynucleotide encoding a dimerization domain, a selectable marker, a polynucleotide encoding a purification sequence and combinations thereof.

16. The polynucleotide of claim 15, wherein the polynucleotide comprises the sequence of one of SEQ ID NO: 2, 6 and 7.

17. The polynucleotide of claim 15, wherein the heavy chain comprises a heavy chain of a 3H9 antibody.

18. The polynucleotide of claim 15, wherein the light chain comprises a light chain of a 3H9 antibody.

19. The polynucleotide of claim 15, wherein the dimerization domain comprises a leucine zipper.

20. The polynucleotide of claim 19, wherein the leucine zipper is selected from the group consisting of a c-fos leucine zipper, a c-jun leucine zipper and combinations thereof.

21. The polynucleotide of claim 15, wherein the purification sequence is selected from the group consisting of the B domain of a protein A, a histidine tag and combinations thereof.

22. A method of identifying an apoptotic cell, the method comprising:
(a) contacting an antibody composition adapted to recognize an eptiope on the surface of an apoptotic cell with a cell; and
(b) detecting association of the antibody composition with the epitope, the association being indicative of an apoptotic cell.

23. The method of claim 22, wherein the antibody composition further comprises an scFv.

24. The method of claim 23, wherein the scFv comprises the amino acid sequence encoded by a nucleic acid sequence comprising one of SEQ ID NOs: 6 and 7.

25. The method of claim 23, wherein the scFv comprises a functional fragment of the antigen binding domain of an scFv.

26. The method of claim 23, wherein the scFv comprises a 3H9 variant.

27. The method of claim 26, wherein the 3H9 variant comprises one or more mutations selected from the group consisting of R53G, I57T, D65G, D56R and S76R.

28. The method of claim 22, wherein the antibody composition further comprises a leucine zipper amino acid sequence.

29. The method of claim 28, wherein the leucine zipper comprises a c-jun leucine zipper.

30. The method of claim 28, wherein the leucine zipper comprises a c-fos leucine zipper.

31. The method of claim 22, wherein the antibody composition further comprises a purification sequence.

32. The method of claim 31, wherein the purification sequence comprises the B domain sequence of protein A.

33. The method of claim 31, wherein the purification sequence comprises a histidine tag.

34. The method of claim 23, wherein the scFv comprises a detectable moiety.

35. The method of claim 34, wherein the detectable moiety comprises a fluorescent label.

36. The method of claim 34, wherein the detectable moiety comprises a radioactive label.

37. The method of claim 34, wherein the detectable moiety comprises an EM radiation-absorbing label.

38. The method of claim 23, wherein the scFv is a monomer, dimer or oligomer.

39. The method of claim 22, wherein the detecting comprises performing an ELISA assay.

40. The method of claim 39, wherein the ELISA is a cellular ELISA.

41. The method of claim 22, wherein the detecting comprises FACS.

42. The method of claim 22, wherein the detecting comprises immunofluorescence microscopy.

43. The method of claim 22, wherein the detecting comprises identifying the presence of a radioemission.

44. The method of claim 22, wherein the detecting comprises performing one or more spectroscopic measurements.

45. The method of claim 22, further comprising determining an amount of cells undergoing apoptosis, based on the detected quantity.

46. The method of claim 22, further comprising treating the cells with an apoptotic-modulating compound before the contacting.

47. The method of claim 46, wherein the apoptotic-modulating compound is selected from the group consisting of staurosporine, camptothecin, or a murine anti-Fas monoclonal antibody.

48. The method of claim 47, wherein the murine anti-Fas monoclonal antibody is 7C11.

49. A method of evaluating the efficacy of a candidate therapeutic compound adapted to effect a change in apoptosis, the method comprising:
(a) contacting an antibody composition adapted to recognize an epitope on the surface of an apoptotic cell with a first sample comprising cells capable of apoptosis;
(b) quantifying an extent to which apoptosis is occurring in the first sample;
(c) contacting a candidate therapeutic with a second sample comprising cells capable of apoptosis;
(d) contacting the antibody composition with the second sample;
(e) quantifying a second degree to which apoptosis is occurring; and
(f) comparing the first and second degrees of apoptosis, whereby the efficacy of a candidate therapeutic compound adapted to effect a change in apoptosis is evaluated.

50. The method of claim 49, wherein the antibody composition further comprises an scFv.

51. The method of claim 50, wherein the scFv comprises a 3H9 variant.

52. The method of claim 51, wherein the 3H9 variant comprises one or more mutations selected from the group consisting of R53G, I57T, D65G, D56R and S76R.

53. The method of claim 49, wherein the quantifying comprises identifying an amount of the antibody composition associated with the cells.

54. The method of claim 49, wherein the quantifying comprises FACS.

55. The method of claim 49, wherein the quantifying comprises performing an ELISA assay.

56. The method of claim 55, wherein the ELISA is a cellular ELISA.

57. The method of claim 49, wherein the quantifying comprises immunofluorescence microscopy.

58. The method of claim 49, wherein the comparing comprises performing a statistical analysis.

59. A kit for detecting apoptotic cells, the kit comprising:
(a) an antibody composition that specifically recognizes an epitope on the surface of an apoptotic cell;
(b) a cell culture medium; and
(c) a detection reagent adapted to indicate the presence of an immunocomplex comprising an antibody composition and an apoptotic cell.

60. The kit of claim 59, wherein the antibody composition comprises an scFv.

61. The kit of claim 60, wherein the scFv comprises one or more of a variable segment of an antibody heavy chain, a variable segment of an antibody light chain, a linker sequence, a dimerization domain, a purification sequence, and combinations thereof.

62. The kit of claim 61, wherein the heavy chain comprises a heavy chain of a 3H9 antibody.

63. The kit of claim 61, wherein the light chain comprises a light chain of a 3H9 antibody.

64. The kit of claim 62 or 63, wherein the 3H9 antibody comprises one or more mutations selected from the group consisting of R53G, I57T, D65G, D56R and S76R.

65. The kit of claim 61, wherein the dimerization domain is a leucine zipper.

66. The kit of claim 65, wherein the leucine zipper is one or more of a c-fos leucine zipper, a c-jun leucine zipper and combinations thereof.

67. The kit of claim 61, wherein the purification sequence is selected from the group consisting of the B domain of a protein A, a histidine tag and combinations thereof.

68. The kit of claim 61, wherein the scFv comprises a dimer.

69. The kit of claim 59, wherein the epitope comprises phosphatidylserine, dioleoyl phosphatidylserine, β2GPI, a nucleoprotein, a constituent of an apoptotic cell surface and combinations thereof.

70. The scFv of claim 69, wherein the epitope is located in a region of a bleb formed on the surface of an apoptotic cell.

71. The kit of claim 59, wherein the medium comprises RPMI 1640 comprising 10% FBS.

72. The kit of claim 59, wherein the detection reagent comprises a moiety selected from the group consisting of a radiolabel, a fluorescent label, a chemiluminescent label and an enzyme.

73. A method of generating an scFv adapted to detect cells undergoing apoptosis, the method comprising:
(a) providing one or more polynucleotide sequences selected from the group consisting of polynucleotide encoding a variable segment of a heavy chain of an antibody, polynucleotide encoding a variable segment of a light chain of an antibody, polynucleotide encoding a linker sequence and polynucleotide encoding a dimerization domain and a purification sequence;
(b) ligating the one or more sequences of DNA into a vector to form an expression vector; and
(c) expressing a protein encoded by a sequence of the expression vector, whereby an scFv adapted to detect cells undergoing apoptosis is generated.

74. The method of claim 73, wherein the one or more polynucleotide sequences comprises a 3H9 variant.

75. The method of claim 74, wherein the 3H9 variant comprises one or more mutations selected from the group consisting of R53G, I57T, D65G, D56R, S76R, and combinations thereof.

76. The method of claim 73, wherein the purification sequence is one or more of the B domain of a protein A and a his tag.

77. The method of claim 73, wherein the dimerization domain is a leucine zipper.

78. The method of claim 77, wherein the leucine zipper is selected from the group consisting of a c-fos leucine zipper and a c-jun leucine zipper.

79. The method of claim 73, wherein the vector comprises a pET26b+ polynucleotide sequence.

80. The method of claim 73, wherein the vector comprises a T7 promoter.

81. The method of claim 73, wherein the vector comprises a selectable marker.

82. The method of claim 73, wherein the expression vector comprises the nucleotide sequence of SEQ ID NO: 2.

83. The method of claim 73, wherein the expressing comprises:
(a) transforming viable bacterial cells with the vector to form transformed cells;
(b) incubating the transformed cells in a suitable growth medium for a desired period of time;
(c) lysing the transformed cells; and
(d) purifying an expressed protein.

84. The method of claim 83, wherein the bacterial cells are E. coli cells.

85. The method of claim 83, wherein the purifying comprises column chromatography.

86. The method of claim 85, wherein the column chromatography is metal-chelation chromatography.

87. The method of claim 85, wherein the column chromatography is size exclusion chromatography.

88. The method of claim 85, wherein the column chromatography is IgG agarose affinity chromatography.

89. A method of screening a population of antibodies to identify an antibody adapted to detect cells undergoing apoptosis, the method comprising:
(a) providing a library comprising one of a population of diverse antibodies and a phage display library comprising an antibody fusion protein to be screened;
(b) contacting the library with a population of cells comprising apoptotic cells to thereby form a mixture;
(c) contacting the mixture with a 3H9-derived antibody composition adapted to specifically recognize an epitope on the surface of an apoptotic cell, the epitope being detectable in cells undergoing apoptosis and undetectable in cells not undergoing apoptosis to thereby form a detection mixture comprising bound antibodies;
(d) contacting the detection mixture with a detectably labeled antibody adapted to recognize the 3H9-derived antibody composition, thereby identifying the presence of apoptotic cells;
(e) separating apoptotic cells from non-apoptotic cells; and
(f) eluting bound antibodies.

90. The method of claim 89, wherein the population of antibodies comprises a single chain variable fragment (scFv).

91. The method of claim 90, wherein the scFv comprises the amino acid sequence of one of SEQ ID NOs: 6 and 7.

92. The method of claim 90, wherein the scFv comprises one or more of a variable segment of an antibody heavy chain, a variable segment of an antibody light chain, a linker sequence, a dimerization domain, a purification sequence and combinations thereof.

93. The method of claim 92, wherein the heavy chain comprises a heavy chain of a 3H9 antibody.

94. The method of claim 92, wherein the light chain comprises a light chain of a 3H9 antibody.

95. The method of claim 92, wherein the dimerization domain is a leucine zipper.

96. The method of claim 95, wherein the leucine zipper is one or more of a c-fos leucine zipper, a c-jun leucine zipper and combinations thereof.

97. The method of claim 92, wherein the purification sequence is selected from the group consisting of the B domain of a protein A, a histidine tag and combinations thereof.

98. The method of claim 89 wherein the epitope is present in a complex comprising phosphatidylserine, dioleoyl phosphatidylserine, β2GPI, a nucleoprotein, a constituent of an apoptotic cell surface and combinations thereof.

99. The method of claim 89, wherein the epitope is located in a region of a bleb formed on the surface of an apoptotic cell.

100. The method of claim 89, wherein the separating is performed by FACS.

101. The method of claim 89, wherein the population of diverse antibodies is derived from a hydriboma fusion.

102. An isolated antibody composition, comprising a region that specifically recognizes an epitope on the surface of an apoptotic cell, the epitope being present in a complex comprising phosphatidylserine, dioleoyl phosphatidylserine, β2GPI, a nucleoprotein, a constituent of an apoptotic cell surface and combinations thereof, and being detectable in cells undergoing apoptosis and undetectable in cells not undergoing apoptosis.


SEQUENCE LISTING (11 sequences)

SEQ ID NO: 1 - protein, 15 aa, Artificial
  misc_feature: synthesized linker sequence
GGGGSSGGGSGGGGS

SEQ ID NO: 2 - DNA, 5319 bp, Artificial
  misc_feature: plasmid construct comprising scFV
atccggccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggttcgagtgcggccgcccatggccatcgccggctgggcagcgaggagcagcagaccagcagcagcggtcggcagcaggtatttcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 3 - DNA, 27 bp, Mus musculus
  misc_feature (1)..(27): k is g or t/u w is a or t/u
tctgaggactccggaakgtattwctgt

SEQ ID NO: 4 - DNA, 29 bp, Mus musculus
atccctgagctcacggtgactgaggttcc

SEQ ID NO: 5 - DNA, 18 bp, Mus musculus
caggggccagtgcataga

SEQ ID NO: 6 - DNA, 1062 bp, Artificial
  misc_feature: mutant construct
ccatggttcaactgcagcagtccggacctgagctggtgaagcctggggcctcagtgaagatttcctgcaaggtttctggctatgcattcagtagctcctggatgaactgggtgaagcagaggcctggaaagggtcttgagtggattggacggatttatcctggagatggagatactaattacaatgggaagttcaagggcaaggccacactgactgcagacaaatcctccagcacagcctacatgcaactcagcagcctgacatctgaggactctgcggtctacttctgtgcaagagcgaggagtaaatattcctatgttatggactactggggtcaagggacctcagtcaccgtgagctccggtggaggaggttcctcaggtggtggatcgggtcggggtggatccgaaaatgtgctgacccagtctccagcaatcatggccgcatctccaggggagaaggtcaccatcacctgcagtgccagctcaagtgtaagttctggtaactttcactggtatcagcagaagccaggctcttctcccaaactctggatttataggacatctaacctggcttctggagtccccgctcgcttcagtggcagtgggtctgggacctcttactctcttacaatcagcagcatggaggccgaagatgctgccacttattactgccagcagtggtgtggttacccattcacgttcggcacggggacaaaattggagatctcgggaggtggaaacgctcggctagaggaaaaagtgaaaaccttgaaagcgcaaaactccgagctggcatccacggccaacatgctcagggaacaggtggcacagcttaagcagaaagtcatgaaccactcgagtgatccgaaagctgacaacaaattcaacaaagaacaacaaaatgctttctatgaaatcttacatttacctaacttaaatgaagaacaacgcaatggtttcatccagtctctgaaagatgatccaagccaaagcgctaaccttttagcagaagctaaaaagctaaatgatgcacaagcaccaaaagctagcttgcggccgc

SEQ ID NO: 7 - DNA, 1062 bp, Artificial
  misc_feature: mutant construct
ccatggttcaactgcagcagtccggacctgagctggtgaagcctggggcctcagtgaagatttcctgcaaggtttctggctatgcattcagtagctcctggatgaactgggtgaagcagaggcctggaaagggtcttgagtggattggacggatttatcctagagatggacgtattaattacaatgggaagttcaaggacaaggccacactgactgcagacaaatcctccagaacagcctacatgcaactcagcagcctgacatctgaggactctgcggtctacttctgtgcaagagcgaggagtaaatattcctatgttatggactactggggtcaagggacctcagtcaccgtgagctccggtggaggaggttcctcaggtggtggatcgggtcggggtggatccgaaaatgtgctgacccagtctccagcaatcatggccgcatctccaggggagaaggtcaccatcacctgcagtgccagctcaagtgtaagttctggtaactttcactggtatcagcagaagccaggctcttctcccaaactctggatttataggacatctaacctggcttctggagtccccgctcgcttcagtggcagtgggtctgggacctcttactctcttacaatcagcagcatggaggccgaagatgctgccacttattactgccagcagtggtgtggttacccattcacgttcggcacggggacaaaattggagatctcgggaggtggaaacgctcggctagaggaaaaagtgaaaaccttgaaagcgcaaaactccgagctggcatccacggccaacatgctcagggaacaggtggcacagcttaagcagaaagtcatgaaccactcgagtgatccgaaagctgacaacaaattcaacaaagaacaacaaaatgctttctatgaaatcttacatttacctaacttaaatgaagaacaacgcaatggtttcatccagtctctgaaagatgatccaagccaaagcgctaaccttttagcagaagctaaaaagctaaatgatgcacaagcaccaaaagctagcttgcggccgc

SEQ ID NO: 8 - DNA, 303 bp, Mus musculus
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccg

SEQ ID NO: 9 - DNA, 321 bp, Mus musculus
  misc_feature (1)..(321): n is g, a, t or c
tgatgacccagactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttnacatgttcnattnacgttcggctnggggacaaagttga

SEQ ID NO: 10 - DNA, 316 bp, Mus musculus
  misc_feature (1)..(316): n is g, a, t or c
agtctccatcctncctggctgtgtcagcaggagagaaggtcactatgagctgcaaatccagtcagagtctgctcacagtagaacccgaaagaagctacttggcttggtaccagcagaaaccagggcagtctcctaaactgctgatctactgggcatccactagggaatctggggtccctgatnncttnacaggcagtggatctgggacagatttnactctcaccatcagcagtgtgcaggctgaagacctggcagtttattactgcaagcaatcttataatctattcacgttcggctnggggacaaagttggaaat

SEQ ID NO: 11 - DNA, 295 bp, Mus musculus
gatgacccagactccagcctccctatctgcatctgtgggagaaactgtcaccatcacatgtcgagcaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtactccattcacgttcggcggacaa